(19) United States
(12) Patent Application Publication
(10) Pub. No.: US 2008/0262506 A1
(43) Pub. Date: Oct. 23, 2008

(54) CATHETER

(75) Inventors: Patrick Griffin, Castlegar (IE); Brendan Casey, Barna (IE); Avril O'Higgins, Renmore (IE); Michael Gilvarry, Ballina (IE); Siora Coll, Roscommon Town (IE)

Correspondence Address: SUGHRUE MION, PLLC, 2100 PENNSYLVANIA AVENUE, N.W., SUITE 800, WASHINGTON, DC, 20037, US

(73) Assignee: SALVIAC LIMITED, Dublin (IE)

(21) Appl. No.: 11/841,259

(22) Filed: Aug. 20, 2007

Related U.S. Application Data
(63) Continuation of application No. 10/180,973, filed on Jun. 27, 2002, now abandoned.
(60) Provisional application No. 60/312,791, filed on Aug. 17, 2001.
(60) Provisional application No. 60/301,820, filed on Jul. 2, 2001.
(60) Provisional application No. 60/341,271, filed on Dec. 20, 2001.
(60) Provisional application No. 60/341,275, filed on Dec. 20, 2001.

(30) Foreign Application Priority Data
Jun. 27, 2001 (IE) 2001-0591
Aug. 20, 2001 (IE) 2001-0772
Dec. 20, 2001 (IE) 2001-1094

(51) Int. Cl. A61M 25/00

(52) U.S. Cl. 606/108

(57) ABSTRACT

A retrieval catheter 1 for retrieval of an embolic protection filter from a vasculature. The catheter 1 comprises a proximal hypotube 3, a radially offset distal shaft 4 and a centering shank 9. A guidewire lumen 13 is provided through the shank 9 in communication with a larger guidewire lumen 7 through the shaft 4 for a continuous guidewire passageway to accommodate passage of a guidewire 21 through the shank 9, the shaft 4 and out through a proximal guidewire opening 6 to facilitate rapid exchange of the catheter 1 over a guidewire 21. The shank 9 is movable between an introduction configuration in which the shank 9 protrudes distally from the shaft 4 to centre the catheter 1 during advancement through a vasculature, and a retrieval configuration in which the shank 9 is located within the guidewire lumen 7 to facilitate retrieval of an embolic protection filter into the shaft 4.

This application is a continuation of Ser. No. 10/180,973 filed Jun. 27, 2002 which claims benefit of priority under 35 U.S.C. § 119 to Irish Patent Application No. 2001/0772, filed Aug. 20, 2001; U.S. Provisional Patent Application No. 60/312,791, filed Aug. 17, 2001; Irish Patent Application No. 2001/1094, filed Dec. 20, 2001; U.S. Provisional Patent Application No. 60/341,275, filed Dec. 20, 2001; Irish Patent Application No. 2001/0591, filed Jun. 27, 2001; and U.S. Provisional Patent Application No. 60/301,820, filed Jul. 2, 2001; all of the disclosures of which are hereby incorporated by reference in their entirety.

### BACKGROUND OF THE INVENTION

### Field of the Invention

This invention relates to a retrieval catheter for retrieving a medical device from a vasculature into the catheter.

In particular this invention relates to a retrieval catheter which is configured to be exchanged over a guidewire in an over-the-wire manner.

This invention also relates to a retrieval catheter, which is configured to be exchanged over a guidewire in a rapid exchange manner during both introduction of the catheter through a vasculature and retrieval of a medical device into the catheter.

Exchange of a catheter over a guidewire using a rapid exchange arrangement enables an interventional procedure to be performed by a single operator in a fast, efficient manner.

The invention is aimed at providing a retrieval catheter for retrieval of a medical device into the catheter.

### SUMMARY OF THE INVENTION

According to the invention there is provided a retrieval catheter comprising:

a catheter shaft defining a reception space for a medical device;

a centering member movable relative to the catheter shaft between an introduction configuration in which the centering member extends distally of the catheter shaft, and a retrieval configuration in which the centering member is located proximally of a distal end of the catheter shaft for retrieval of a medical device into the reception space; and

an operating element coupled to the centering member to facilitate movement of the centering member between the introduction configuration and the retrieval configuration;

along at least a portion of the length of the operating element, the cross-sectional area of the operating element being small relative to the cross-sectional area of the catheter shaft.

The centering member extends distally of the catheter shaft to centre the retrieval catheter during advancement through the vasculature, and in particular during crossing of a lesion or a treatment device located in the vasculature proximally of the medical device being retrieved. In this way, the possibility of snagging of the catheter on the treatment device and/or dislodgement of embolic material at the lesion is minimized.

The centering member is retractable independently of retrieval of a filter into the reception space prior to filter retrieval. Therefore the force to retract the centering member is not added to the retraction force of the filter.

The centering member can be used to re-deploy the filter if the clinician decides that further procedures are required proximal to the filter. If the centering member was not controllable, a new filter would have to be delivered to the site and then deployed at the site. To achieve this the lesion site would have to be crossed by a new filter delivery system increasing risk of dislodging emboli and increasing procedure time.

If the catheter snags on a stent the user can manipulate the relative positions of the centering member and the catheter shaft from the proximal end to help free the catheter.

The ability to adjust the relative position of the centering member and the catheter shaft allows the user to adjust the stiffness profile of the catheter distal end. This ability to tailor the distal stiffness profile can aid in stent or lesion crossing and in navigation through tortuous anatomy. It also allows the user to employ a centering member with an increased transition length for trackability reasons, but prior to filter retrieval this can be partially retracted to reduce the parking space required proximal to the filter.

In one embodiment of the invention a guidewire opening is provided in the catheter shaft, the guidewire opening being located a substantial distance distally of a proximal end of the catheter for rapid exchange of the catheter over a guidewire.

The retrieval catheter of the invention is adapted for rapid exchange over a guidewire. This rapid exchange arrangement enables the catheter to be passed by a single user through a vasculature over the guidewire quickly and efficiently, without requiring assistance.

In a preferred case the cross-sectional area of the operating element is small relative to the cross-sectional area of the catheter shaft in the region of the guidewire opening. Ideally in the introduction configuration the cross-sectional area of the operating element is small relative to the cross-sectional area of the catheter shaft for a distance of at least 10 mm distally of the guidewire opening. Most preferably in the introduction configuration the cross-sectional area of the operating element is small relative to the cross-sectional area of the catheter shaft for a distance of at least 20 mm distally of the guidewire opening. Desirably in the introduction configuration the cross-sectional area of the operating element is small relative to the cross-sectional area of the catheter shaft for a distance of at least 30 mm distally of the guidewire opening. In a particularly preferred case in the introduction configuration the cross-sectional area of the operating element is small relative to the cross-sectional area of the catheter shaft for a distance of at least 40 mm distally of the guidewire opening.

The diameter of the operating element may be in the range of from 0.008″ to 0.015″. Ideally the diameter of the operating element is in the range of from 0.01″ to 0.012″.

In another embodiment of the invention in the retrieval configuration the centering member is located in the reception space. Preferably in the retrieval configuration the centering member is configured to fit in the reception space distally of the guidewire opening.

The centering member does not occlude the opening in the catheter shaft when the centering member is in the introduction configuration or in the retrieval configuration. This allows unobstructed rapid exchange of the retrieval catheter over a guidewire throughout the procedure, and in particular during both introduction of the catheter through the vasculature and during retrieval of a medical device into the catheter.

The centering member may be slidably movable relative to the catheter shaft between the introduction configuration and the retrieval configuration.

In one case the centering member comprises a coiled spring. Preferably the centering member comprises longitudinal stiffening means. Ideally the longitudinal stiffening means comprises a shaft extending through at least part of the centering member.

The centering member may comprise an atraumatic distal tip. Preferably the tip tapers distally inwardly.

The centering member may define a guidewire lumen therethrough. Preferably the guidewire lumen of the centering member is movable relative to the guidewire opening in the catheter shaft upon movement of the centering member between the introduction configuration and the retrieval configuration.

In a further embodiment the operating element is attached to the centering member. The operating element may be integral with at least part of the centering member. In one preferred case the operating element is rotatably attached to the centering member. Ideally the operating element comprises a ring rotatably received in a groove on the centering member.

In another embodiment the centering member extends distally of the operating element.

The operating element may comprise a control wire. Preferably the operating element comprises a pull wire.

The operating element may comprise a coiled spring. The operating element may comprise a fiber. The operating element may comprise a hypotube. The operating element may be at least partially of a polymeric material.

In one case the operating element defines a lumen therethrough.

In a preferred case the catheter comprises two or more operating elements. Ideally the operating elements are braided together.

In another embodiment of the invention the catheter shaft comprises a proximal shaft portion and a distal pod, the pod at least partially defining the reception space. The proximal shaft portion may be offset in the radial direction from the pod. Preferably the proximal shaft portion is of a smaller diameter than the pod.

In one case the catheter shaft comprises a mounting piece for attaching the pod to the proximal shaft portion. Preferably the distal end of the proximal shaft portion is located distally of the proximal end of the pod. Ideally the mounting piece is more flexible than the proximal shaft portion and the pod.

In another case the mounting piece is more stiff than the proximal shaft portion and the pod.

The mounting piece may taper proximally inwardly.

The mounting piece may taper distally inwardly.

In a preferred case the guidewire opening in the catheter shaft is provided by an opening in the mounting piece. Ideally the guidewire opening faces in a direction substantially parallel to the longitudinal axis of the catheter. Most preferably the guidewire opening faces proximally.

In a further embodiment the catheter shaft comprises means to guide passage of a guidewire through the guidewire opening in the catheter shaft. The guide means ensures a smooth, controlled passage of a guidewire through the guidewire opening in the catheter shaft. The guide means may comprise a guide ramp. In one case the ramp is funnel-shaped.

The guide means may comprise at least one guide tube through which a guidewire may pass. Preferably the guide tube extends at least partially internally through the catheter shaft. The guide tube may extend at least partially externally of the catheter shaft. Ideally the guide tube is mounted to the catheter shaft. The guide tube may be mounted to the centering member.

In one case the catheter comprises two or more guide tubes movable relative to one another in a telescoping manner.

In one case the invention provides a retrieval catheter for retrieving an embolic protection filter from a vasculature.

In another case the invention provides a retrieval catheter for retrieving a stent from a vasculature.

In another aspect of the invention there is provided a catheter comprising a proximal shaft portion and a distal shaft portion attached to the proximal shaft portion, and means to stiffen the catheter at the junction between the proximal shaft portion and the distal shaft portion.

In one embodiment of the invention the catheter comprises a mounting piece for attaching the distal shaft portion to the proximal shaft portion. Preferably the distal end of the proximal shaft portion is located distally of the proximal end of the distal shaft portion to stiffen the junction. Ideally the mounting piece is more flexible than the proximal shaft portion and the distal shaft portion.

In another case the mounting piece is more stiff than the proximal shaft portion and the distal shaft portion to stiffen the junction.

The catheter may comprise strain relief means. Preferably the mounting piece tapers distally inwardly. Ideally the mounting piece tapers proximally inwardly.

A guidewire opening may be provided in the catheter, the guidewire opening being located a substantial distance distally of a proximal end of the catheter for rapid exchange of the catheter over a guidewire. Preferably the guidewire opening is provided by an opening in the mounting piece. Ideally the guidewire opening faces in a direction substantially parallel to the longitudinal axis of the catheter.

In one case the catheter comprises means to guide passage of a guidewire through the guidewire opening in the catheter. The means to guide passage may be provided by the mounting piece.

According to a further aspect, the invention provides a retrieval catheter comprising:

a catheter shaft defining a reception space for a medical device;

a guidewire opening being provided in the catheter shaft; and

a centering member movable relative to the catheter shaft between an introduction configuration in which the centering member extends distally of the catheter shaft, and a retrieval configuration in which the centering member is located proximally of a distal end of the catheter shaft for retrieval of a medical device into the reception space;

the centering member defining a guidewire lumen therethrough;

the guidewire lumen of the centering member being movable relative to the guidewire opening in the catheter shaft upon movement of the centering member between the introduction configuration and the retrieval configuration.

In one embodiment of the invention the guidewire opening in the catheter shaft is located a substantial distance distally of a proximal end of the catheter for rapid exchange of the catheter over a guidewire. Preferably the guidewire opening in the catheter shaft faces in a direction substantially parallel to the longitudinal axis of the catheter.

The catheter may comprise means to guide passage of a guidewire through the guidewire opening in the catheter shaft.

In one case the catheter comprises an operating element coupled to the centering member to facilitate movement of the centering member between the introduction configuration and the retrieval configuration.

Ideally the centering member is slidably movable relative to the catheter shaft.

The invention also provides in another aspect a retrieval catheter comprising:

a catheter shaft defining a reception space for a medical device;

a guidewire opening being provided in the catheter shaft located a substantial distance distally of a proximal end of the catheter for rapid exchange of the catheter over a guidewire;

centering member movable relative to the catheter shaft between an introduction configuration in which the centering member extends distally of the catheter shaft, and a retrieval configuration in which the centering member is located proximally of a distal end of the catheter shaft for retrieval of a medical device into the reception space; and

an operating element coupled to the centering member to facilitate movement of the centering member between the introduction configuration and the retrieval configuration;

a distal end of the centering member and a proximal end of the centering member translating relative to the catheter shaft during movement of the centering member relative to the catheter shaft.

In one embodiment along at least a portion of the length of the operating element, the cross-sectional area of the operating element is small relative to the cross-sectional area of the catheter shaft.

Preferably in the retrieval configuration the centering member is located in the reception space.

According to another aspect of the invention, there is provided a method of retrieving a medical device from a vasculature, the method comprising the steps of:

providing a retrieval catheter comprising a catheter shaft and a centering member;

advancing the catheter through a vasculature over a guidewire in a rapid-exchange manner, with the centering member extending from a distal end of the catheter shaft, until the centering member is proximally adjacent to the medical device to be retrieved;

manipulating the centering member from externally of the vasculature to move the centering member proximally relative to the catheter shaft;

retrieving the medical device into the catheter shaft; and

withdrawing the retrieval catheter and the retrieved medical device from the vasculature over the guidewire in a rapid exchange manner.

In one case the guidewire is advanced through the vasculature before inserting the retrieval catheter into the vasculature.

In another case the guidewire remains in the vasculature during withdrawal of the retrieval catheter from the vasculature.

It will be appreciated that in the retrieval catheter of the invention a variety of means may be employed to facilitate movement of the centering member proximally relative to the catheter shaft for retrieval.

### BRIEF DESCRIPTION OF THE DRAWINGS

The invention will be more clearly understood from the following description of some embodiments thereof, given by way of example only, with reference to the accompanying drawings, in which:

FIG. 1 is a perspective view of a retrieval catheter according to the invention passing over a guidewire;

FIG. 2 is a partially cross-sectional, side view of the catheter of FIG. 1;

FIGS. 2(a) and 2(b) are enlarged, cross-sectional, side views of parts of the catheter of FIG. 2;

FIG. 3 is a perspective view of a part of the catheter of FIG. 2;

FIG. 4 is a cut-away, perspective view of the part of FIG. 3;

FIG. 5 is a perspective view of another part of the catheter of FIG. 2;

FIG. 6 is a cut-away, perspective view of the part of FIG. 5;

FIGS. 6(a) to 6(c) are partially cross-sectional, side views of the catheter of FIG. 2, in use;

FIGS. 6(d) and 6(e) are cross-sectional, side views of the catheter of FIG. 2, in use;

FIG. 6(f) is a cross-sectional, side view of another retrieval catheter, in use;

FIG. 6(g) is an enlarged, cross-sectional, side view of the catheter of FIG. 6(f);

FIG. 7 is a partially cut-away, perspective view of another retrieval catheter according to the invention passing over a guidewire;

FIG. 7(a) is a cross-sectional, side view of another retrieval catheter according to the invention;

FIGS. 8 and 9 are partially cut-away, perspective views of other retrieval catheters according to the invention;

FIGS. 9(a) and 9(b) are cross-sectional, side views of parts of other retrieval catheters according to the invention;

FIG. 9(c) is a perspective view of a part of a further retrieval catheter according to the invention;

FIG. 9(d) is a perspective view of a portion of the part of FIG. 9(c);

FIG. 9(e) is a perspective view of a part of another retrieval catheter according to the invention;

FIG. 10 is a partially cut-away, perspective view of another retrieval catheter according to the invention;

FIG. 11 is an enlarged, cross-sectional, side view of a part of the catheter of FIG. 10;

FIG. 11(a) is a perspective view of the part of FIG. 11;

FIG. 11(b) is a cross-sectional, side view of the part of FIG. 11;

FIG. 11(c) is a view along line XI-XI in FIG. 11(b);

FIG. 12 is a cut-away, perspective view of another retrieval catheter according to the invention passing over a guidewire;

FIG. 13 is a partially cut-away, perspective view of the catheter of FIG. 12, in use;

FIG. 13(a) is a cross-sectional, side view of a further retrieval catheter according to the invention;

FIG. 13(b) is a cross-sectional, side view of the catheter of FIG. 13(a), in use;

FIG. 14 is a partially cut-away, perspective view of another retrieval catheter according to the invention;

FIG. 15 is a partially cut-away, perspective view of another retrieval catheter according to the invention passing over a guidewire;

FIG. 16 is a partially cut-away, perspective view of the catheter of FIG. 15, in use;

FIGS. 17 and 18 are views similar to FIGS. 15 and 16 of another retrieval catheter according to the invention;

FIGS. 18(a) and 18(b) are cross-sectional, side views of other retrieval catheters according to the invention;

FIG. 18(c) is a partially cross-sectional, side view of another retrieval catheter according to the invention passing over a guidewire;

FIG. 18(d) is a partially cross-sectional, side view of the catheter of FIG. 18(c), in use;

FIG. 19 is a partially cut-away, perspective view of a further retrieval catheter according to the invention;

FIGS. 20 and 21 are partially cut-away, perspective views of other retrieval catheters according to the invention;

FIG. 22 is a cut-away, perspective view of another retrieval catheter according to the invention;

FIG. 23 is a cut-away, perspective view of the catheter of FIG. 22, in use;

FIG. 23(a) is a cross-sectional, side view of another retrieval catheter according to the invention;

FIG. 23(b) is a cross-sectional, side view of the catheter of FIG. 23(a), in use;

FIG. 24 is a partially cut-away, perspective view of another retrieval catheter according to the invention;

FIG. 25 is a cross-sectional, side view of another retrieval catheter according to the invention passing over a guidewire;

FIG. 26 is a cross-sectional, side view of the catheter of FIG. 25, in use;

FIGS. 27 and 28 are views similar to FIGS. 25 and 26 of another retrieval catheter according to the invention;

FIGS. 29 and 30 are views similar to FIGS. 25 and 26 of a further retrieval catheter according to the invention;

FIG. 31 is a partially cut-away, perspective view of a further retrieval catheter according to the invention;

FIG. 32 is a partially cut-away, perspective view of a part of another retrieval catheter according to the invention;

FIG. 33 is a partially cut-away, perspective view of another retrieval catheter according to the invention;

FIG. 34 is a partially cut-away, perspective view of an end of the catheter of FIG. 33;

FIG. 35 is a cut-away, perspective view of a part of the catheter of FIG. 34;

FIG. 36 is a schematic view illustrating formation of the part of FIG. 35;

FIG. 37 is a cross-sectional, side view of another retrieval catheter according to the invention;

FIG. 38 is a cross-sectional, side view of the catheter of FIG. 37, in use;

FIGS. 39 to 42 are cross-sectional, side views of a part of other retrieval catheters according to the invention;

FIG. 43 is a cross-sectional, side view of the part of FIG. 42, in use;

FIG. 44 is a partially cut-away, perspective view of another retrieval catheter according to the invention;

FIG. 45 is a partially cut-away, perspective view of the catheter of FIG. 44, in use;

FIG. 46 is a partially cut-away, perspective view of another retrieval catheter according to the invention passing over a guidewire;

FIG. 47 is another partially cut-away, perspective view of the catheter of FIG. 46 passing over a guidewire;

FIG. 48 is a view along line I-I in FIG. 47;

FIGS. 49 and 50 are partially cut-away, perspective views of another retrieval catheter according to the invention passing over a guidewire;

FIG. 51 is a partially cut-away, perspective view of a further retrieval catheter according to the invention passing over a guidewire;

FIG. 52 is a cross-sectional, side view of another retrieval catheter according to the invention;

FIG. 53 is a cross-sectional, side view of a part of the catheter of FIG. 52, in use;

FIGS. 54 to 56 are cross-sectional, side views of a part of other retrieval catheters according to the invention passing over a guidewire;

FIG. 57 is a cross-sectional, side view of another retrieval catheter according to the invention;

FIG. 58 is a view along line XX-XX in FIG. 57;

FIG. 59 is a cross-sectional, side view of the catheter of FIG. 57, in use;

FIG. 60 is an enlarged, perspective view of a part of the catheter of FIG. 59;

FIGS. 61 and 62 are cross-sectional, side views of other retrieval catheters according to the invention passing over a guidewire; and

FIGS. 63 to 70 are cross-sectional, side views of a part of other retrieval catheters according to the invention.

### DETAILED DESCRIPTION OF THE INVENTION

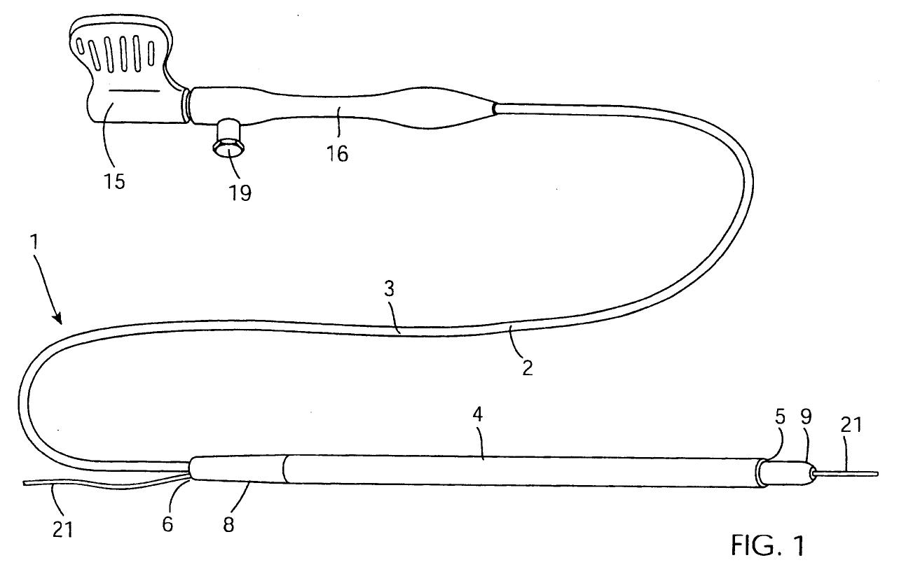

Referring to the drawings and initially to FIGS. 1 to 6(c) thereof, there is illustrated a retrieval catheter 1 according to the invention for retrieval of a medical device from a vasculature.

An example of a medical device for which the retrieval catheter 1 of the invention may be employed to retrieve is an embolic protection filter. Filters are generally deployed distally of a treatment site, such as a region of stenosis in a vasculature, prior to carrying out a treatment procedure such as an angioplasty procedure, or an atherectomy procedure, or a stenting procedure, on the stenosed region. The filter collects and retains any dislodged embolic material generated as a result of the treatment procedure, and thereby prevents emboli from migrating distally through the vascular system. After completion of the treatment procedure, the filter with its retained embolic load is retrieved into a catheter and withdrawn from the vasculature.

The catheter 1 comprises a catheter body 2 and a centering means movable relative to the catheter body 2.

The catheter body 2 comprises, in this case, a proximal hypotube 3 and a radially offset distal shaft 4. The catheter body 2 has a distal guidewire opening 5 at the distal end of the shaft 4, and a proximal guidewire opening 6 in the sidewall of the catheter body 2. A guidewire lumen 7 extends between these openings 5,6 for passage of a guidewire 21 through the lumen 7 and out of the lumen 7 through the proximal guidewire opening 6 to facilitate rapid exchange of the catheter 1 over the guidewire 21.

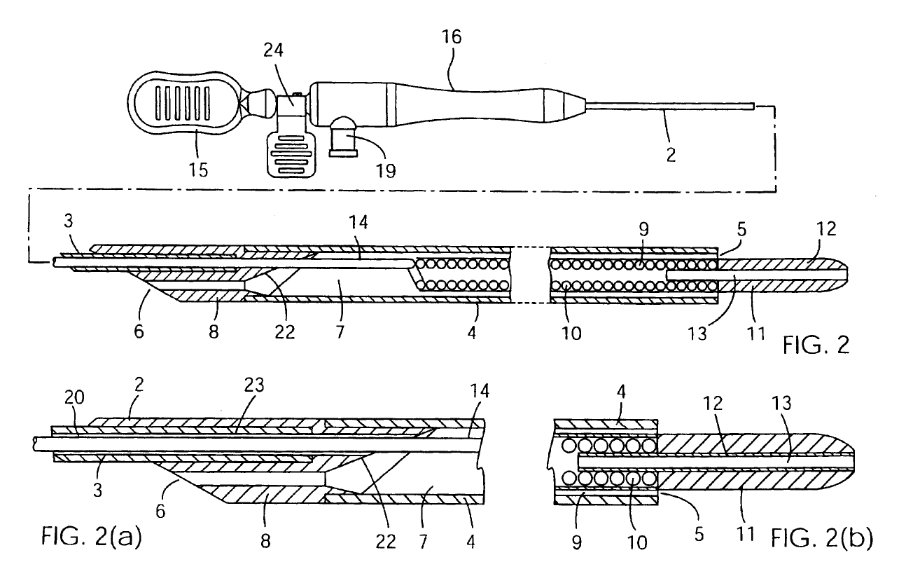

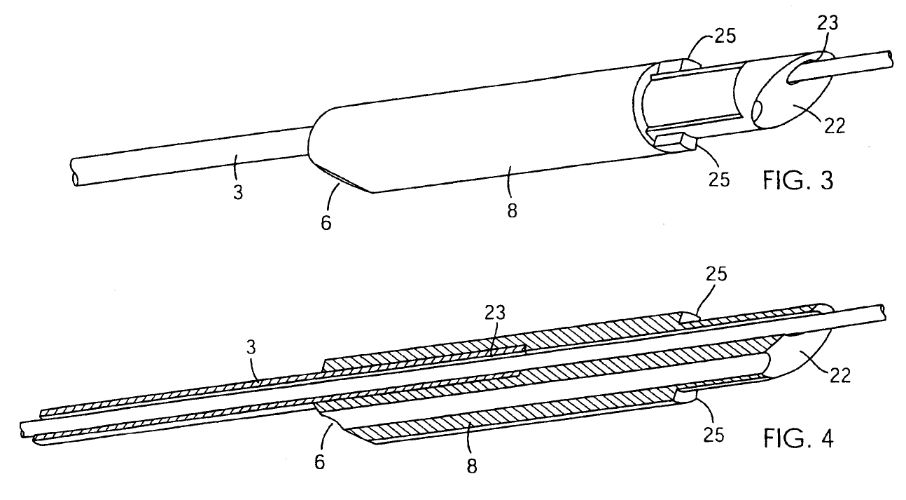

A junction piece 8 is provided to connect the proximal hypotube 3 to the distal shaft 4, the hypotube 3 and the shaft 4 being fixed to the junction piece 8 by means of bonding. A step 25 is provided on the junction piece 8 (FIGS. 3 and 4) for attachment of the shaft 4 to the junction piece 8 with a smooth crossing profile as illustrated in FIG. 2(a).

A variety of means may be used to attach the hypotube 3 and the shaft 4 to the junction piece 8, such as welding, solvent bonding, or insert moulding.

In this case, the proximal guidewire opening 6 is provided by an opening in the junction piece 8 in communication with the guidewire lumen 7 at the proximal end of the shaft 4.

As illustrated in FIGS. 3 and 4, a sloping distal end face 22 of the junction piece 8 slopes towards the proximal guidewire opening 6 in a conical manner. In this manner, the conical face 22 acts to guide the guidewire 21 passing through the guidewire lumen 7 towards the proximal guidewire opening 6 somewhat in the manner of a funnel. The conical face 22 funnels the guidewire 21 through the proximal guidewire opening 6 in at least two planes for enhanced control of passage of the guidewire 21.

As illustrated in FIG. 2, the proximal guidewire opening 6 faces in a direction substantially parallel to the guidewire lumen 7. This arrangement enables the guidewire 21 to pass through the proximal guidewire opening 6 in a direction substantially parallel to the guidewire lumen 7, and in this way the overall profile of the catheter 1 as it is exchanged over the guidewire 21 is kept to a minimum.

The junction piece 8 acts as a bridge to provide a transition in stiffness between the hypotube 3 and the shaft 4.

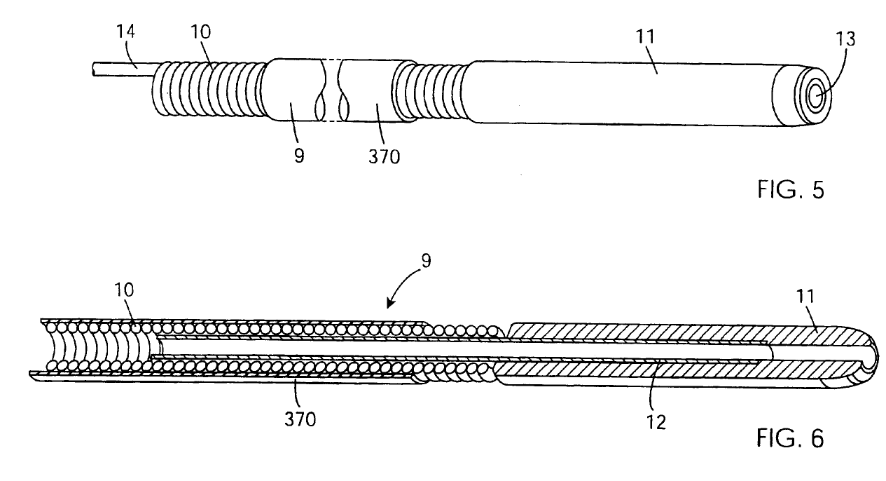

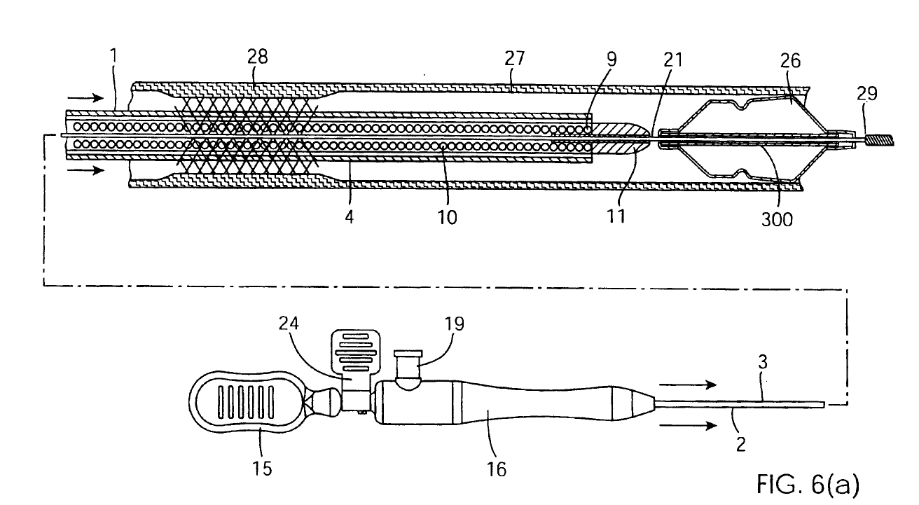

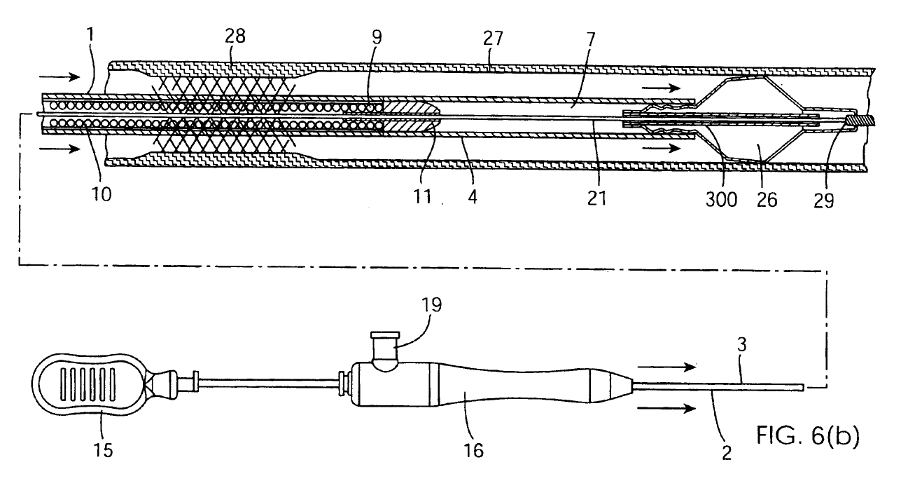

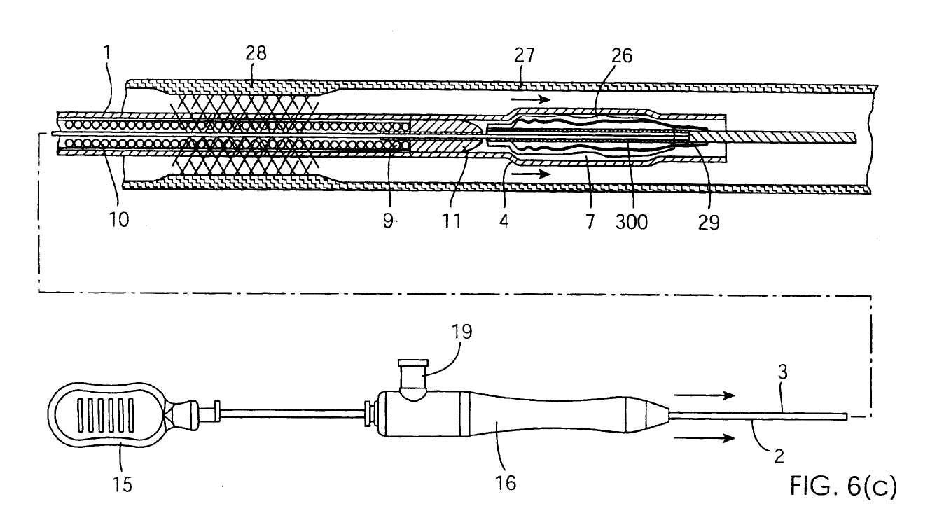

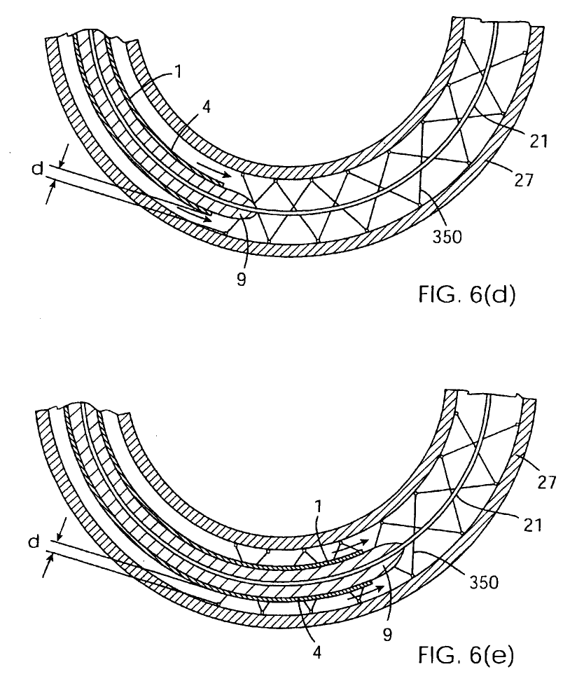

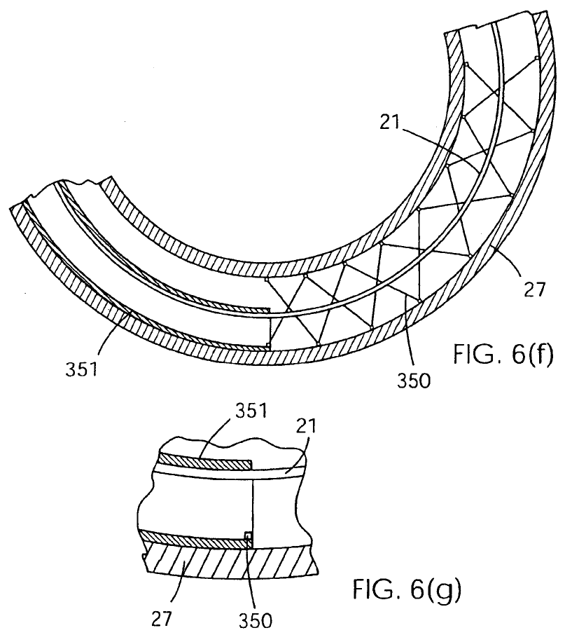

The centering means is provided, in this case, by a shank 9. As illustrated in FIGS. 5 and 6, the shank 9 comprises a proximal coiled spring 10, and a distal compressible tip 11 mounted around a stiff inner shaft 12. A guidewire lumen 13 is provided through the shaft 12 and the coiled spring 10 in communication with the larger guidewire lumen 7 for a continuous guidewire passageway to accommodate passage of the guidewire 21 through the shaft 9 and into the guidewire lumen 7.

The coiled spring 10 has the characteristics of good pushability and good lateral trackability, both of which are important during advancement of the catheter 1 through a vasculature, in particular if the vasculature is tortuous or narrowed. In particular, the coiled spring 10 enhances the buckle resistance of the shaft 4.

The coiled spring 10 also provides radial support to the shaft 4.

The tip 11 tapers distally inwardly similar to an arrow-head shape for a smooth crossing profile for the catheter 1 in the introduction configuration. The tip 11 is of an injection moulded polymeric material, and is bonded to the coiled spring 10.

The inner shaft 12 enhances the longitudinal stiffness of the shank 9 which aids the pushability of the catheter 1. In particular, the shaft 12 provides a transition in stiffness from the soft tip 11 to the stiffer spring 10 to minimize the possibility of buckling of the shank 9.

The shank 9 is movable between an introduction configuration, in which the shank 9 protrudes distally of the distal end of the shaft 4, as illustrated in FIG. 2, and a retrieval configuration, in which the shank 9 is located within the guidewire lumen 7 proximally of the distal end of the shaft 4.

A polytetrafluoroethylene tubing 370 is heat shrunk to the exterior of the coiled spring 10 proximally of the tip 11 to ease movement of the shank 9 relative to the shaft 4.

In the introduction configuration, the protruding shank 9 assists in centering the distal end of the shaft 4 during advancement of the retrieval catheter 1 through a vasculature. In this manner the possibility of snagging of the retrieval catheter 1 during advancement through the vasculature is minimized, even when crossing a treatment site or a deployed medical device. The retrieval catheter 1 is thus particularly suitable for retrieving a medical device from a location in a vasculature distal of a lesion, such as a build-up of atheroma in the vasculature, or distal of a treatment device, such as a stent deployed in the vasculature.

In the retrieval configuration, the distal end opening 5 of the shaft 4 is open to facilitate retrieval of a medical device, such as an embolic protection filter or a stent, from a vasculature into the guidewire lumen 7.

The longitudinal length of the shank 9 is such that in the retrieval configuration the shank 9 fits in the guidewire lumen 7 distally of the proximal guidewire opening 6. This configuration ensures that the proximal guidewire opening 6 is not occluded in the retrieval configuration by the shank 9, and passage of the guidewire 21 through the proximal guidewire opening 6 is not interfered with in any way by the movement of the shank 9 from the introduction configuration to the retrieval configuration. The catheter 1 may thus be passed over the guidewire 21 in a rapid exchange manner in both the introduction configuration and the retrieval configuration.

The guidewire lumen 7 also acts as a reception space into which a medical device, such as an embolic protection filter, may be retrieved.

An elongate actuator 14 extends distally from the proximal end of the catheter body 2 through an actuator lumen 20 in the hypotube 3, through an aligned lumen 23 in the junction piece 8 (FIGS. 3 and 4), through the guidewire lumen 7 to the shank 9, to which the actuator 14 is coupled. In this case, the actuator 14 is in the form of a wire integral with the coiled spring 10 (FIG. 5). By moving the actuator 14 proximally relative to the catheter body 2 the shank 9 may be moved from the introduction configuration to the retrieval configuration.

Proximal and distal handles 15, 16 are provided for gripping the actuator 14 and the catheter body 2 respectively. The proximal handle 15 is fixed to the actuator 14, and the distal handle 16 is fixed to the hypotube 3 by means of a polyolefin tube. A flushing luer 19 in the distal handle 16 may be used to flush the actuator lumen 20 in the hypotube 3 and the guidewire lumen 7. A releasable clamping clip 24 is provided to hold the handles 15, 16 in a fixed position relative to one another during flushing of the catheter 1 and during introduction of the catheter 1 through a vasculature.

The actuator 14 enables a user to accurately control the position of the shank 9 relative to the catheter body 2 by manipulating the actuator 14 from a location externally of the vasculature. In this way, the shank 9 may be moved independently of the medical device being retrieved.

In addition, should the shank 9 become incorrectly positioned during performance of an intravascular procedure, for example snagged on a stent, the actuator 14 enables a user to correctly reposition the shank 9 as desired from a location externally of the vasculature.

The shank 9 may be constructed with a relatively low compressive strength because the action of pulling the actuator wire 14 to move the shank 9 proximally relative to the catheter body 2 does not subject the shank 9 to compressive stresses. By pulling the shank 9 proximally into the guidewire lumen 7, the shank 9 will be subjected to tensile stresses.

The junction piece 8 tapers inwardly proximally. Because the shank 9 is always located distally of the proximal guidewire opening 6, this enables the proximal hypotube portion 3 of the catheter body 2 to be constructed with a lower profile.

A low-friction coating, such as of polytetrafluoroethylene, may be provided around the catheter body 2 to ease passage of the catheter body 2 through a vasculature.

The distal portion of the shaft 4 comprises a radially expandable body with at least one, and in this case two, reinforcement columns extending longitudinally along the expandable body. The columns and the expandable body are integrally formed, for example by a co-extrusion process, with the columns extending partially within the expandable body.

The radially expandable body facilitates retrieval of the medical device into the guidewire lumen 7. The reinforcement columns prevent buckling of the shaft 4, for example during the retrieval procedure when longitudinal retrieval forces are exerted on the shaft 4 by the medical device being retrieved, or during advancement of the catheter 1 through a vasculature.

The distal portion of the shaft 4 may be mounted to the proximal portion of the shaft 4, for example by means of bonding, such as with an adhesive, or by fusing, such as by welding.

The distal portion of the shaft 4 is similar to that described in our International patent application No. PCT/IE02/00048, the relevant contents of which are incorporated herein by reference.

The proximal portion of the shaft 4 is similar to that described in our International patent application No. PCT/IE02/00047, the relevant contents of which are incorporated herein by reference.

The retrieval catheter 1 of the invention is suitable for retrieving a medical device, such as an embolic protection filter from a vasculature. A filter 26 is typically deployed in a vasculature 27 mounted on a guidewire 21 downstream of a treatment region in the vasculature, such as a stenosis 28, prior to treatment of the stenosis 28, such as by a stenting procedure or by an angioplasty procedure. Any emboli released during the treatment are captured and safely retained in the filter 26 and thus migration of embolic material downstream, which could otherwise have potentially harmful consequences, is prevented. When the treatment of the vasculature 27 has been completed, the deployed filter 26 with retained embolic material therein is retrieved as follows.

The proximal end of the guidewire 21, which extends proximally from the vasculature 27, is inserted into the shaft 12 of the shank 9, threaded through the small guidewire lumen 13, then through the large guidewire lumen 7, guided towards the proximal guidewire opening 6 by the conical face 22, and out through the proximal guidewire opening 6 in the junction piece 8. With the shank 9 in the protruding introduction configuration, the catheter 1 is advanced over the guidewire 21 through the vasculature 27 in a rapid-exchange manner until the shank 9 crosses the treatment region 28, and the shank 9 is proximally adjacent to the deployed filter 26 in the vasculature 27 (FIG. 6(a)).

The champing clip 24 is removed and the proximal handle 15 is moved proximally while holding the distal handle 16 fixed to move the actuator 14 proximally and maintain the position of the catheter body 2 fixed in the vasculature. In this manner, the shank 9 is moved from the introduction configuration to the retrieval configuration. In this retrieval configuration, the shank 9 is located in the guidewire lumen 7 distally of the proximal guidewire opening 6 in the junction piece 8. Passage of the guidewire 21 from the guidewire lumen 7 through the proximal guidewire opening 6 is thus not occluded or obstructed in any way.

The distal opening 5 is now clear for retrieval of the filter 26 into the guidewire lumen 7. In this case, the filter 26 is moved proximally relative to the catheter 1 by moving the guidewire 21 proximally relative to the catheter 1 to cause an engagement surface 29 on the guidewire 21 to engage with a tubular member 300 of the filter 26, and thereby move the filter 26 proximally relative to the catheter 1.

One such arrangement by which a guidewire may be used to move a medical device proximally relative to a retrieval catheter is described in our International patent application No. PCT/IE01/00052, the relevant contents of which are incorporated herein by reference.

To retrieve the filter 26, the guidewire 21 is retracted until a stop on the guidewire 21 abuts the tubular member 300 of the filter 26. The catheter body 2 is then advanced to gradually collapse the filter 26 and retrieve the filter 26 into the guidewire lumen 7 (FIG. 6(b)) until the collapsed filter 26 is fully retrieved within the guidewire lumen 7 (FIG. 6(c)). The guidewire stop 29 prevents movement of the filter 26 distally during collapse.

The retrieval catheter 1 with the retrieved filter 26 may then be withdrawn from the vasculature 27 over the guidewire 21 in a rapid exchange manner.

Alternatively, the guidewire 21 may be left in place in the vasculature 27 while the retrieval catheter 1 with the retrieved filter 26 is withdrawn from the vasculature 27. This enables further exchange of medical devices over the guidewire 21 after retrieval of the filter 26 and withdrawal of the retrieval catheter 1, for example to perform an angiographic procedure on the vasculature 27.

The centering shank 9 has a smooth crossing profile and helps to keep the sheath 4 centered on the guidewire during advancement. By advancing the retrieval catheter 1 through the vasculature 27 with the shank 9 in the protruding introduction configuration, this ensures that the catheter 1 is centered during the advancement with a clearance d between the sheath 4 and a stent 350, as illustrated in FIGS. 6(d) and 6(e). In this manner, the possibility of snagging of the retrieval catheter 1 on a treatment device, such as the stent 350, and/or dislodgement of embolic material from a lesion is minimized.

For a retrieval catheter 351 without a centering member, there is a likelihood of the open distal end of the catheter 351 snagging on a strut of the stent 350 as the catheter 351 is advanced through the vasculature 27, as illustrated in FIGS. 6(f) and 6(g), in particular when the vasculature 27 is tortuous or narrow.

The filter 26 may alternatively be retrieved into the guidewire lumen 7 by retracting the guidewire 21 to abut the stop on the guidewire 21 with the tubular member 300 of the filter 21 and pull the filter 21 proximally into the guidewire lumen 7 of the catheter body 2 while holding the catheter body 2 in a fixed position.

It will be appreciated that the filter 26 may alternatively be retrieved into the guidewire lumen 7 by any suitable movement of the catheter 1 distally relative to the deployed filter 26.

A seal may be provided around the coiled spring 10, for example in the form of a polymeric tubing, to facilitate flushing of the guidewire lumen 13 of the shank 9.

In an alternative embodiment, the arrow-head shaped tip 11 may be omitted. In such a case, the coiled spring 10 may taper inwardly distally at the distal end for a smooth crossing profile.

The junction piece 8 may be bonded or moulded or welded or fused to the shaft 4 and/or to the hypotube 3.

Use of the retrieval catheter 1 for retrieval of an embolic filter has been described above by way of example only. It will be appreciated that the retrieval catheter 1 is also suitable for retrieval of medical devices other than embolic protection filters from a vasculature.

It will further be appreciated that the retrieval catheter 1 has been described configured for rapid exchange over a guidewire by means of example only. The retrieval catheter according to the invention is also suitable for over-the-wire exchange over a guidewire.

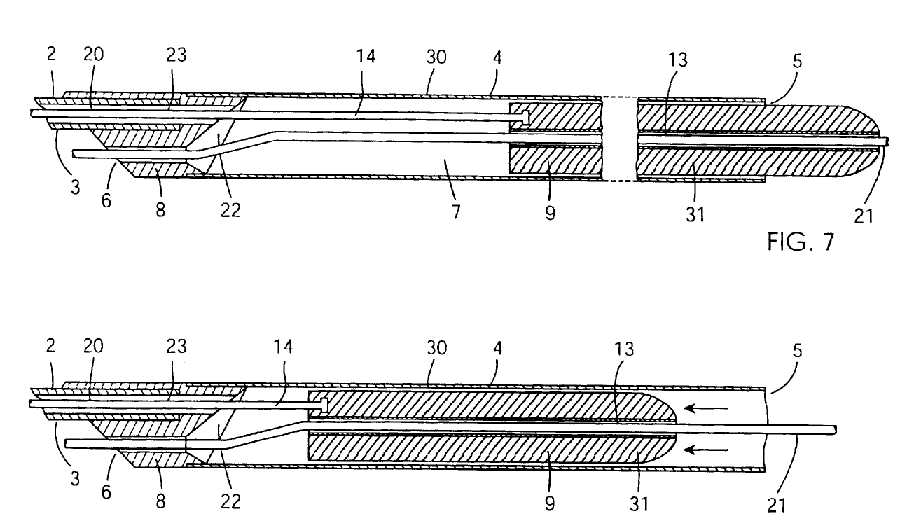

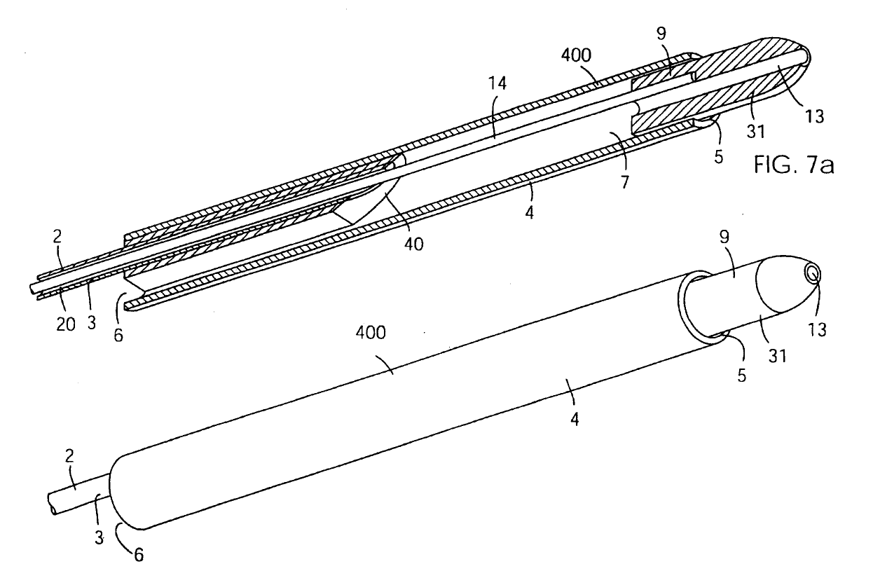

FIG. 7 illustrates another retrieval catheter 30 according to the invention, which is similar to the retrieval catheter 1 of FIGS. 1 to 6(c), and similar elements in FIG. 7 are assigned the same reference numerals.

In this case, the shank 9 comprises a unitary polymeric member 31 tapering distally inwardly. The actuator wire 14 is fixedly attached to the polymeric member 31.

The location of the shank 9 in the retrieval configuration in the guidewire lumen 7 is illustrated in dashed lines in FIG. 7. In this retrieval configuration, the shank 9 fits in the guidewire lumen 7 distally of the proximal guidewire opening 6. The catheter 30 is thus passable over the guidewire 21 in a rapid exchange manner in the retrieval configuration.

Referring to FIG. 7(a) there is illustrated another retrieval catheter 400 according to the invention, which is similar to the retrieval catheter 30 of FIG. 7, and similar elements in FIG. 7(a) are assigned the same reference numerals.

In this case the shaft 4 is directly attached to the hypotube 3 with the proximal end of the shaft 4 overlapping the distal end of the hypotube 3. No junction piece is provided between the hypotube 3 and the shaft 4.

A distal end face 401 of the hypotube 3 is sloped to assist in guiding passage of a guidewire from the guidewire lumen 7 out through the proximal guidewire opening 6.

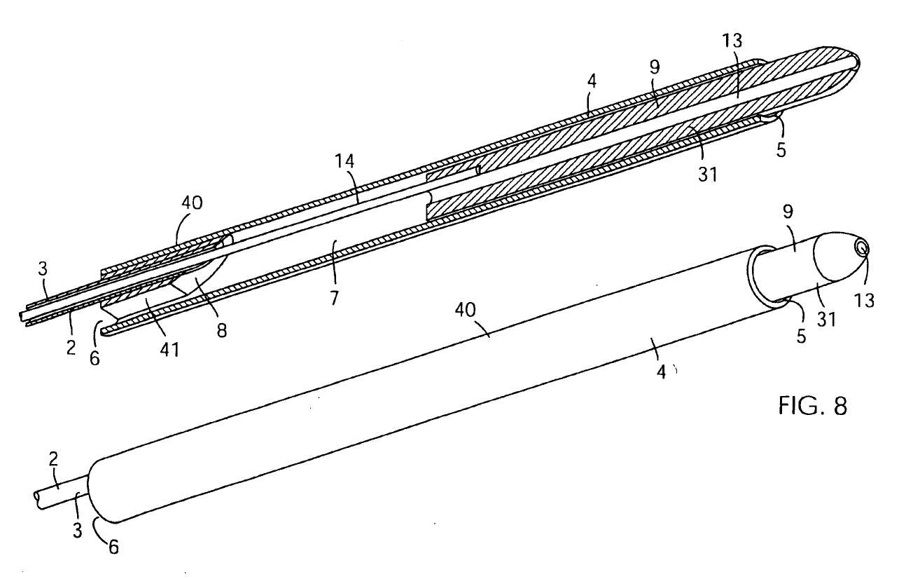

In FIG. 8 there is illustrated another retrieval catheter 40 according to the invention, which is similar to the retrieval catheter 30 of FIG. 7, and similar elements in FIG. 8 are assigned the same reference numerals.

In this case, the junction piece 8 is substantially U-shaped to define a U-shaped channel 41 connecting the guidewire lumen 7 in communication with the proximal guidewire opening 6. The channel 41 assists in guiding passage of the guidewire 21 from the guidewire lumen 7 out through the proximal guidewire opening 6 in a smooth manner.

The longitudinal length of the shank 9 is such that in the retrieval configuration the shank 9 fits in the guidewire lumen 7 distally of the proximal guidewire opening 6 to enable rapid exchange of the retrieval catheter 40 over the guidewire 21 in both the introduction configuration and the retrieval configuration.

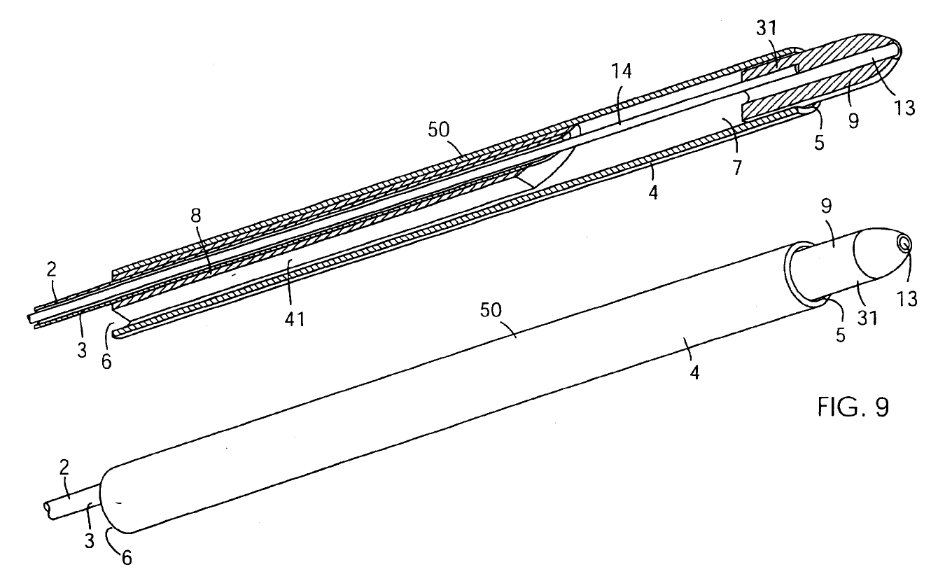

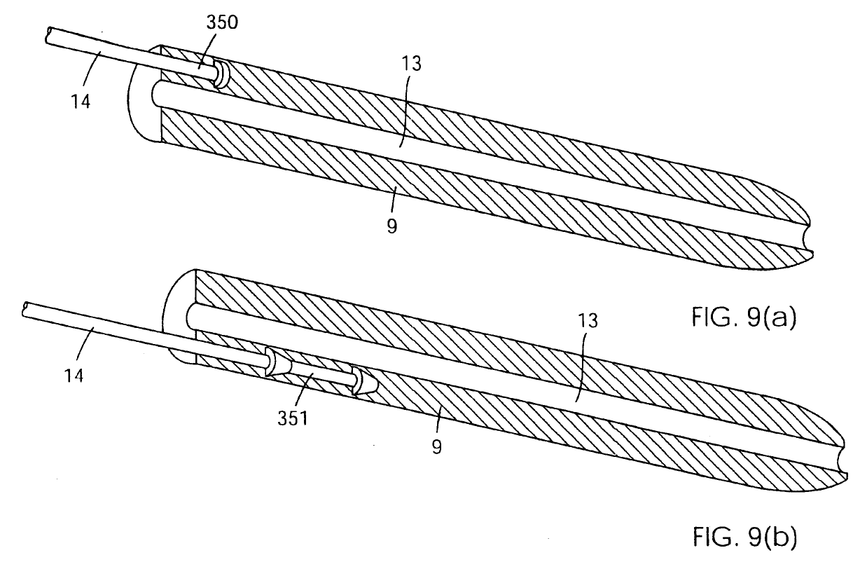

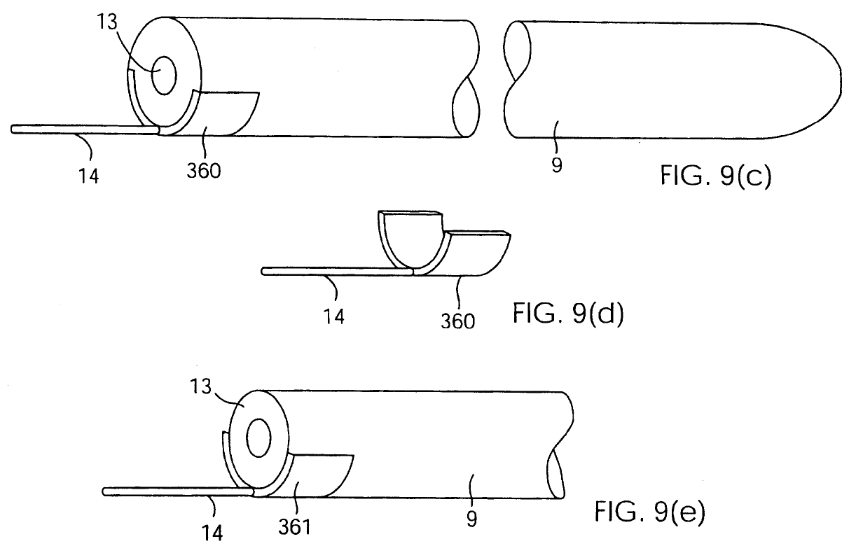

However it will be appreciated that the longitudinal length of the shank 9 and/or of the junction piece 8 may be varied to suit the clinical procedure being performed. For example, in the retrieval catheter 50 of FIG. 9, the shank 9 is longitudinally shorter and the junction piece 8 is longitudinally longer than in the retrieval catheter 40 of FIG. 9. This arrangement results in a stiffer catheter in the region of the proximal guidewire opening 6 and a more trackable catheter towards the distal end of the shaft 4. Thus it may be seen that by suitable modifications of the retrieval catheter of the invention, a range of performance characteristics may be achieved depending on the clinical procedure desired to be performed.

The actuator 14 may be fixedly attached to the shank 9 by any suitable means. For example a mechanical lock 350 may be formed at the distal end of the actuator 14, as illustrated in FIG. 9(a). The shank 9 may then be moulded or bonded over this lock 350 for a secure attachment of the actuator 14 to the shank 9.

The mechanical lock 351 may alternatively have a barbed formation, as illustrated in FIG. 9(b). In this case, the mechanical lock 351 may be pushed into the proximal end of the shank 9 to secure the actuator 14 to the shank 9.

As illustrated in FIGS. 9(c) and 9(d), the actuator 14 may be formed into a curved dish 360 at the distal end of the actuator 14. The flattened configuration of the dish 360 increases the surface area in contact with the shank 9 for a secure attachment of the actuator 14 to the shank 9.

The dish 360 may be recessed into the shank 9 for a smooth crossing profile from the shank 9 to the dish 360 (FIG. 9(c)).

Alternatively the dish 361 may be attached to the shank 9 standing proud of the external surface of the shank 9, as illustrated in FIG. 9(e).

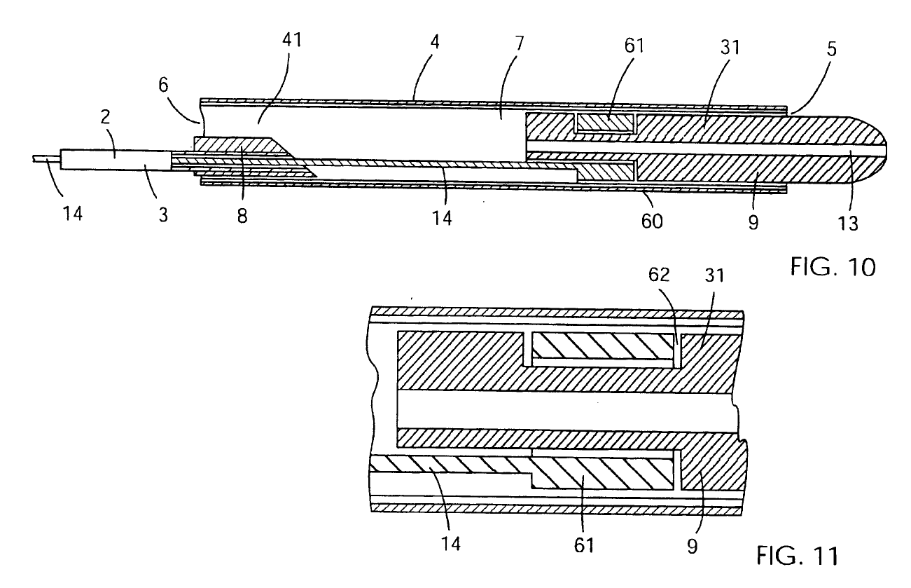

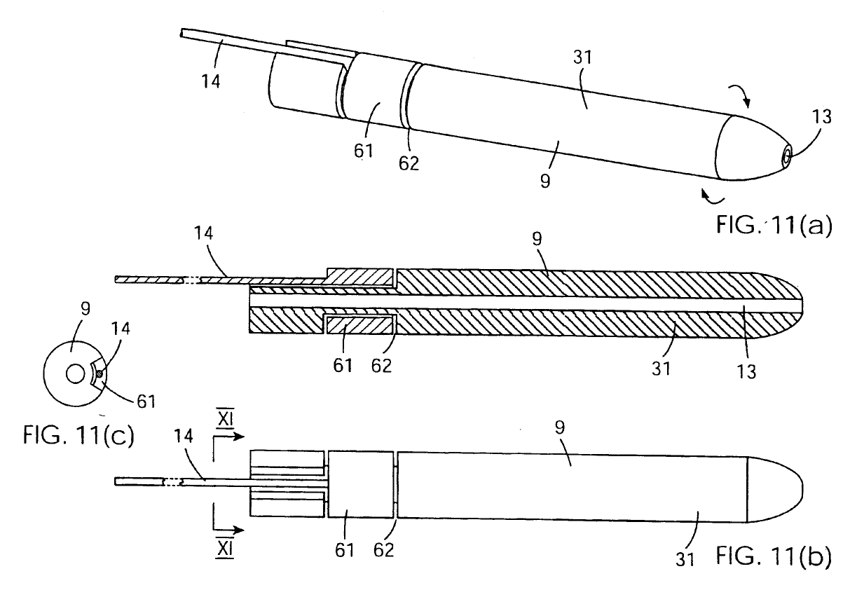

Referring to FIGS. 10 to 11(c), there is illustrated another retrieval catheter 60 according to the invention, which is similar to the retrieval catheter 40 of FIG. 8, and similar elements in FIGS. 10 and 11 are assigned the same reference numerals.

The actuator wire 14 is fixed to a slip ring 61 rotatably held within an annular grove 62 formed on the shank 9. This arrangement facilitates rotation of the actuator 14 relative to the shank 9, as illustrated in FIG. 11(a). Thus the actuator 14 and the guidewire 21 are prevented from becoming entangled or wrapped together.

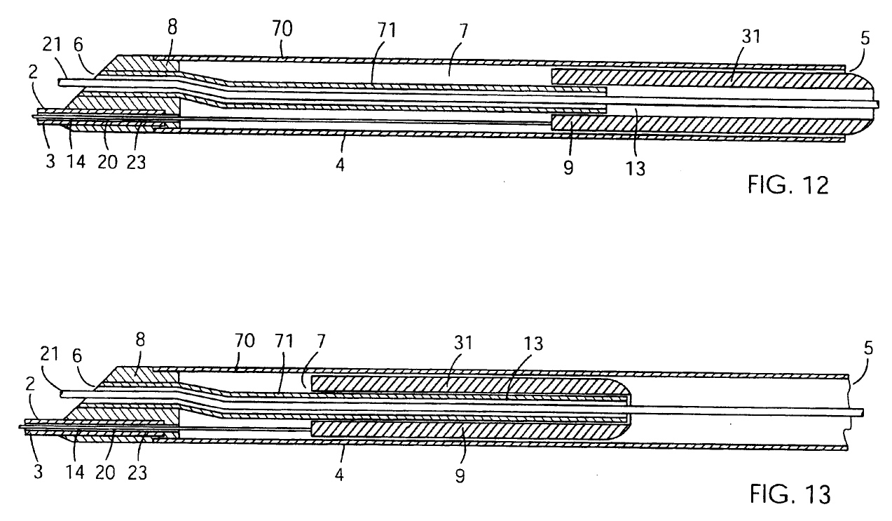

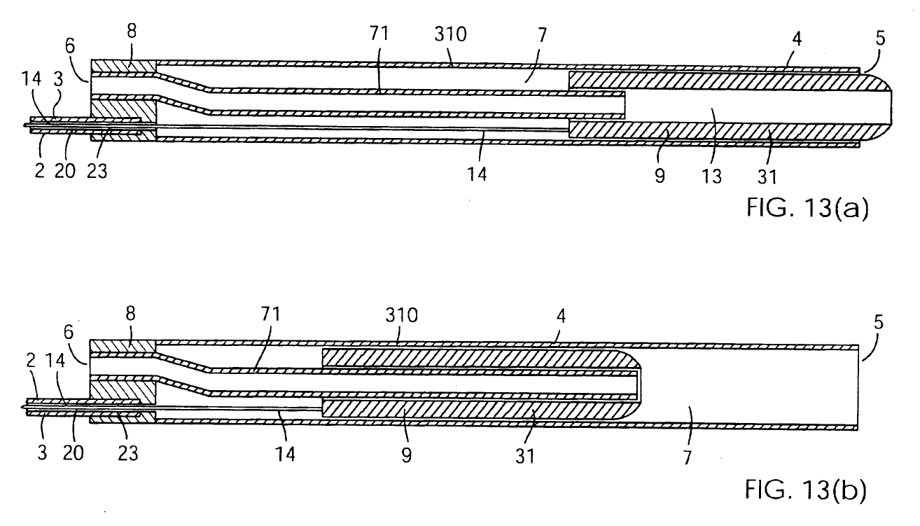

FIGS. 12 and 13 illustrate another retrieval catheter 70 according to the invention, which is similar to the catheter 30 of FIG. 7, and similar elements in FIGS. 12 and 13 are assigned the same reference numerals.

The catheter 70 comprises a guide tube 71 extending distally from the junction piece 8. The guide tube 71 is fixedly attached to the junction piece 8. In both the introduction configuration (FIG. 12) and the retrieval configuration (FIG. 13), the tube 71 extends distally into the guidewire lumen 13 of the shank 9. As the shank 9 moves proximally from the protruding introduction configuration of FIG. 12 to the retracted retrieval configuration of FIG. 13, the shank 9 slides over the tube 71 in a telescoping manner until the distal end of the tube 71 emerges from the distal end of the guidewire lumen 13.

In this position, the distal end of the tube 71 may act as a proximal stop to prevent the medical device being retrieved into the catheter 70 from moving proximally of the tube 71 during the retrieval procedure. This may eliminate the need for radiopacity in the system.

In addition, the guide tube 71 ensures smooth, controlled passage of the guidewire 21 through the proximal guidewire opening 6.

In this case the junction piece 8 does not have a sloping distal end face.

A suitable material for the guide tube 71 is a polymeric material, such as polyurethane, or nylon, or PEBA. Alternatively the guide tube 71 could be provided in the form of a close coiled spring. These materials do not add significantly to the overall stiffness of the retrieval catheter 70.

It will be appreciated that in the retrieval configuration, the distal end of the tube 71 may alternatively be located within the guidewire lumen 13 of the shank 9.

FIGS. 13(a) and 13(b) illustrate another retrieval catheter 310 according to the invention, which is similar to the retrieval catheter 70 of FIGS. 12 and 13, and similar elements in FIGS. 13(a) and 13(b) are assigned the same reference numerals.

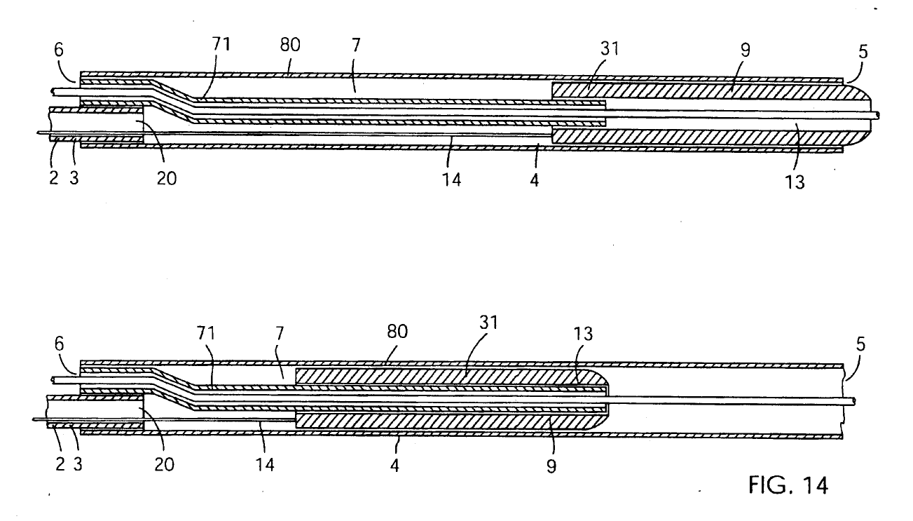

In FIG. 14 there is illustrated a further retrieval catheter 80 according to the invention, which is similar to the retrieval catheter 40 of FIG. 8, and similar elements in FIG. 14 are assigned the same reference numerals.

The catheter 80 comprises the guide tube 71 extending distally from the proximal guidewire opening 6 into the guidewire lumen 13 of the shank 9.

The guide tube 71 is fixed between the distal end of the hypotube 3 and the proximal end of the shaft 4. No junction piece is provided in this case.

In both the introduction configuration and the retrieval configuration the tube 71 extends distally into the guidewire lumen 13 of the shank 9.

The longitudinal length of the shank 9 is relatively large in the catheter 80 of FIG. 14 compared for example to the catheter 70 of FIGS. 12 and 13.

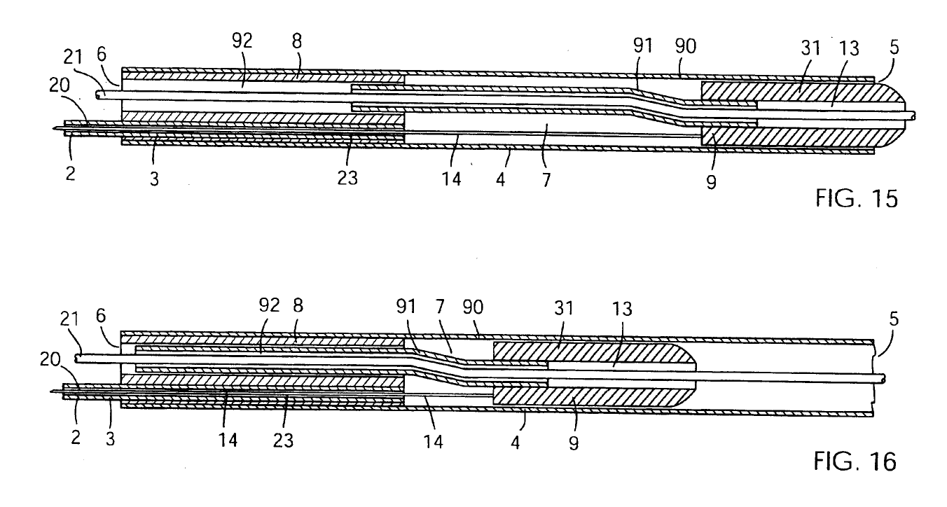

Referring to FIGS. 15 and 16, there is illustrated another retrieval catheter 90 according to the invention, which is similar to the catheter 70 of FIGS. 12 and 13, and similar elements in FIGS. 15 and 16 are assigned the same reference numerals.

In this case, the guide tube 91 is fixedly attached to the shank 9 extending proximally from the shank 9 into the guidewire lumen 92 of the junction piece 8 in both the introduction configuration (FIG. 15) and the retrieval configuration (FIG. 16). As the shank 9 moves from the protruding introduction configuration of FIG. 15 to the retracted retrieval configuration of FIG. 16, the tube 91 slides proximally through the guidewire lumen 92 in the junction piece 8 in a telescoping manner.

This arrangement of the guide tube 91 and the co-operating lumen 92 in the junction piece 8 ensures a smooth, controlled passage of the guidewire 21 out through the proximal guidewire opening 6.

In addition the guide tube 91 prevents tangling or interference between the guidewire 21 and the actuator wire 14.

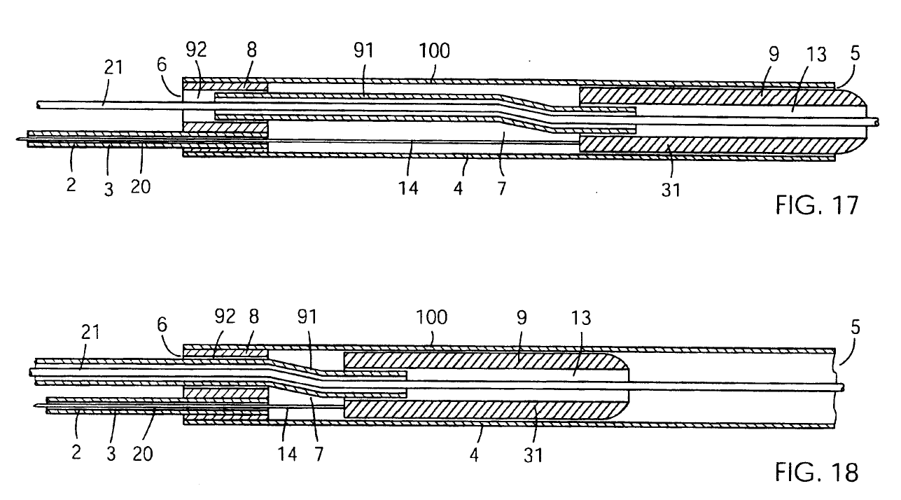

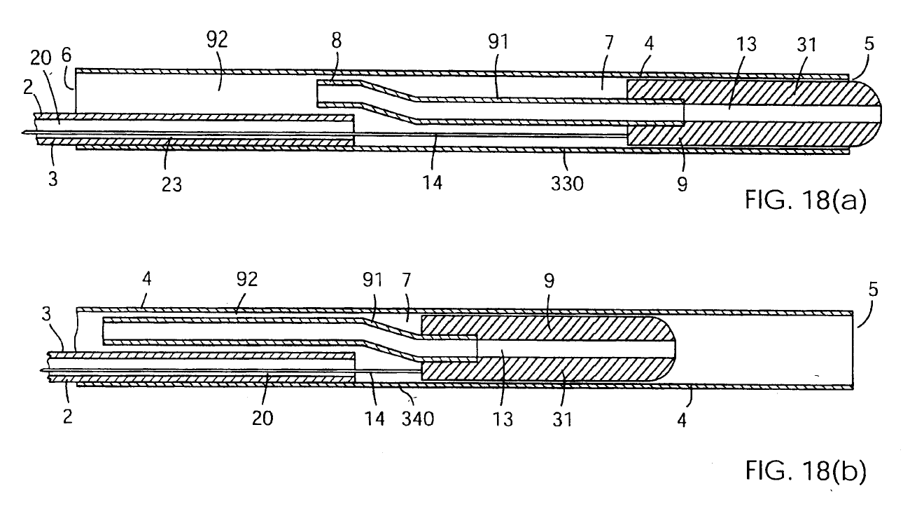

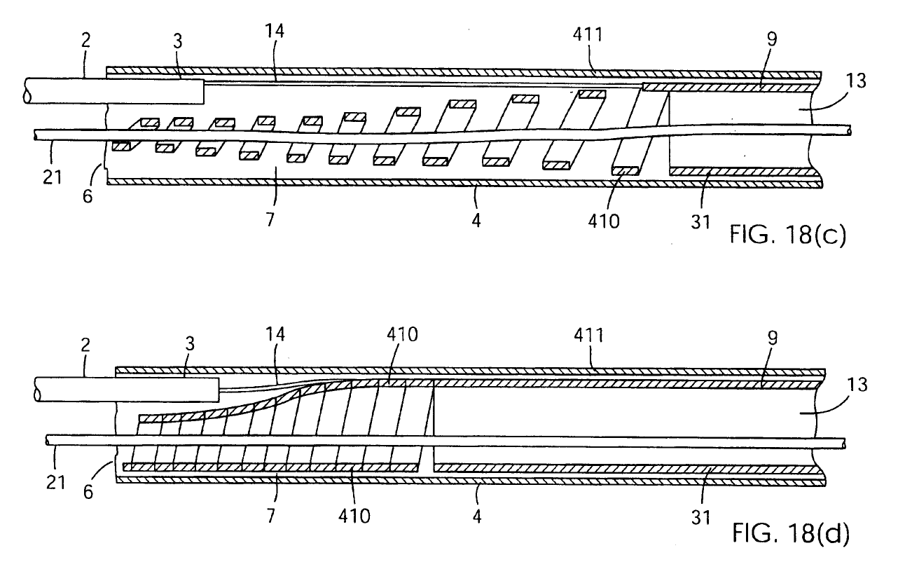

In the retrieval catheter 100 of FIGS. 17 and 18, the junction piece 8 is longitudinally shorter and the shank 9 is longitudinally longer than in the retrieval catheter 90 of FIGS. 15 and 16.

In the introduction configuration, the guide tube 91 extends proximally through the guidewire lumen 92 with the proximal end of the guide tube 91 adjacent the proximal guidewire opening 6.

As the shank 9 is moved from the introduction configuration (FIG. 17) to the retrieval configuration (FIG. 18), the guide tube 91 slides proximally through the guidewire lumen 92. As illustrated in FIG. 18, in the retrieval configuration part of the guide tube 91 extends proximally of the proximal guidewire opening 6 externally of the guidewire lumen 7.

In addition, the relatively long shank 9 provides enhanced pushability for advancement of the catheter 100 through a vasculature.

FIG. 18(a) illustrates another retrieval catheter 330 according to the invention, which is similar to the retrieval catheter 90 of FIGS. 15 and 16, and similar elements in FIG. 18(a) are assigned the same reference numerals.

In the retrieval catheter 340 of FIG. 18(b), the shaft 4 is fixedly attached directly to the hypotube 3 with the proximal end of the shaft 4 overlapping the distal end of the hypotube 3. No junction piece is provided in this case.

Referring to FIGS. 18(c) and 18(d) there is illustrated a further retrieval catheter 411 according to the invention, which is similar to the retrieval catheter 340 of FIG. 18(b), and similar elements in FIGS. 18(c) and 18(d) are assigned the same reference numerals.

In this case the guide tube 410 is in the form of a spiral cut tube. When the shank 9 is in the introduction configuration (FIG. 18(c)), the tube 410 is stretched to extend from the proximal guidewire opening 6 to the proximal end of the member 31. In this way the guidewire 21 is guided from the guidewire lumen 13 of the member 31 through the tube 410, and out through the proximal guidewire opening 6.

When the shank 9 is in the retrieval configuration (FIG. 18(d)), the tube 410 is compressed to guide the guidewire 21 from the guidewire lumen 13 of the member 31 through the proximal guidewire opening 6 without the tube 410 extending out through the proximal guidewire opening 6.

The spiral cut tube 410 provides a highly trackable means of guiding the guidewire 21 from the shank 9 out through the proximal guidewire opening 6. The guidewire 21 is at least partially enclosed by the tube 410 along the full length between the shank 9 and the proximal guidewire opening 6.

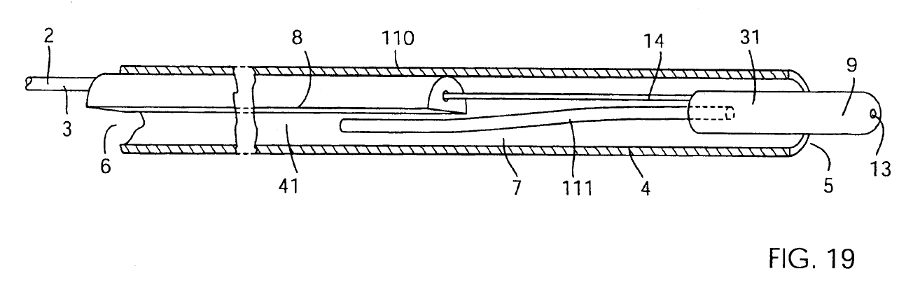

In FIG. 19 there is illustrated another retrieval catheter 110 according to the invention, which is similar to the retrieval catheter 50 of FIG. 9, and similar elements in FIG. 19 are assigned the same reference numerals.

The catheter 110 comprises a guide tube 111 fixedly attached to the shank 9 extending proximally into the U-shaped channel 41 in both the introduction configuration and the retrieval configuration.

As the shank 9 is moved from the introduction configuration to the retrieval configuration, the guide tube 111 slides proximally through the channel 41 in a telescoping manner.

The guide tube 111 assists in a smooth, controlled passage of the guidewire 21 through the channel 41 and out through the proximal guidewire opening 6.

The guide tube 111 also assists in preventing tangling or interference between the guidewire 21 and the actuator wire 14.

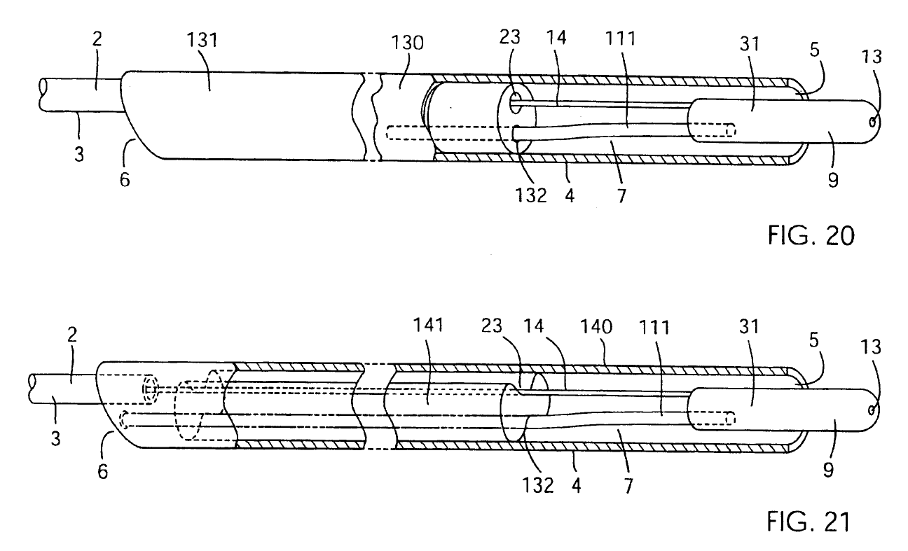

FIG. 20 illustrates another retrieval catheter 130 according to the invention, which is similar to the retrieval catheter 110 of FIG. 19, and similar elements in FIG. 20 are assigned the same reference numerals.

In this case, the catheter 130 comprises a tubular junction piece 131 between the hypotube 3 and the shaft 4. The junction piece 131 has an actuator lumen 23 extending therethrough for passage of the actuator 14 from the lumen 20 of the hypotube 2 through the junction piece 131 distally to the shank 9. The junction piece 131 also has a guidewire lumen 132 extending therethrough to connect the proximal guidewire opening 6 in communication with the guidewire lumen 7.

The guide tube 111 is attached to the proximal end of the shank 9, and extends proximally from the shank 9 into the guidewire lumen 132 of the junction piece 131 in both the introduction configuration and the retrieval configuration.

As the shank 9 moves proximally from the introduction configuration to the retrieval configuration, the guide tube 111 telescopes relative to the junction piece 131 through the guidewire lumen 132. In this manner, passage of the guidewire 21 through the proximal guidewire opening 6 is assisted, and tangling of the actuator wire 14 and the guidewire 21 is avoided.

In FIG. 21 there is illustrated a further retrieval catheter 140 according to the invention, which is similar to the retrieval catheter 130 of FIG. 20, and similar elements in FIG. 21 are assigned the same reference numerals.

The catheter 140 comprises a junction piece 141 shaped to define the actuator lumen 23 and the guidewire lumen 132 between two concave portions of the surface of the junction piece 141 and the interior surface of the shaft 4. The junction piece has a “butterfly” shape.

It will be appreciated that any suitable shape may be employed to define the lumen a between the outer surface of the junction piece and the interior surface of the shaft 4.

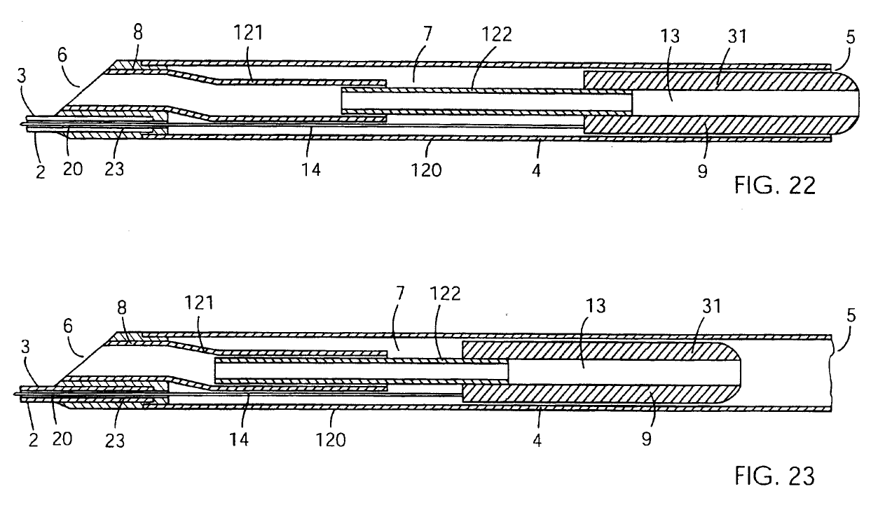

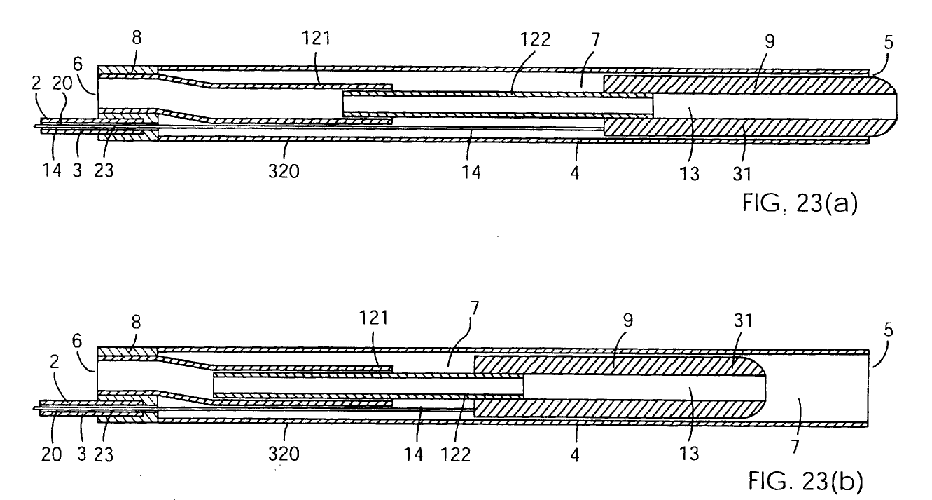

Referring to FIGS. 22 and 23 there is illustrated another retrieval catheter 120 according to the invention, which is similar to the retrieval catheter 70 of FIGS. 12 and 13 and to the retrieval catheter 90 of FIGS. 15 and 16, and similar elements in FIGS. 22 and 23 are assigned the same reference numerals.

In this case, the catheter 120 comprises a first guide tube 121 fixedly attached to the junction piece 8 extending distally, and a second guide tube 122 fixedly attached to the shank 9 extending proximally. In both the introduction configuration (FIG. 22) and the retrieval configuration (FIG. 23), the guide tubes 121, 122 overlap with the tube 122 extending into the tube 121. The tubes 121, 122 are aligned for passage of the guidewire 21 through the tubes 121, 122 and out through the proximal guidewire opening 6.

In use, the tubes 121, 122 slide over one another in an overlapping manner as the shank 9 moves from the protruding introduction configuration, as illustrated in FIG. 22, to the retrieval configuration, as illustrated in FIG. 23.

This arrangement of telescoping tubes 121, 122 guides the passage of the guidewire 21 through the proximal guidewire opening 6, which ensures a smooth, controlled rapid exchange action.

In addition, this arrangement provides a smooth transition by facilitating exit of the guidewire 21 parallel to the shaft 4.

Further the telescoping arrangement prevents the actuator wire 14 and the guidewire 21 from becoming tangled or interfering with each other in any way because the pathways for each wire 14, 21 are isolated from one another.

FIGS. 23(a) and 23(b) illustrate another retrieval catheter 320 according to the invention, which is similar to the retrieval catheter 120 of FIGS. 22 and 23, and similar elements in FIGS. 23(a) and 23(b) are assigned the same reference numerals.

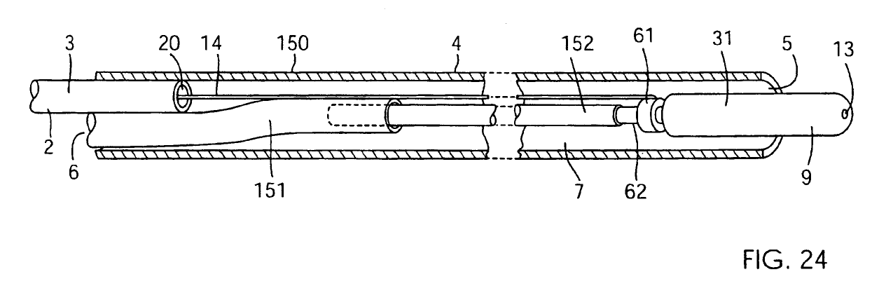

In FIG. 24 there is illustrated another retrieval catheter 150 according to the invention, which is similar to the retrieval catheter 80 of FIG. 14, and similar elements in FIG. 24 are assigned the same reference numerals.

The catheter 150 comprises a first guide tube 151 fixedly attached between the hypotube 3 and the shaft 4 extending distally, and a second guide tube 152 fixedly attached to the shank 9 extending proximally. In both the introduction configuration and the retrieval configuration, the guide tubes 151, 152 overlap with the tube 152 extending into the tube 151. The tubes 151, 152 are aligned for passage of the guidewire 21 through the tubes 151, 152 and out through the proximal guidewire opening 6.

As the shank 9 moves proximally from the introduction configuration to the retrieval configuration, the tubes 151, 152 slide over one another in an overlapping manner.

The actuator wire 14 is rotatably attached to the shank 9 by means of the ring 61 and the groove 62, in a manner similar to that described previously with reference to FIGS. 10 and 11.

The shank 9 comprises a distal nose 202 of a relatively hard material at the distal end of the relatively soft polymeric member 31. During proximal movement of the medical device being retrieved into the reception space 7, the medical device engages the nose 202 to push the shank 9 proximally from the introduction configuration to the retrieval configuration.

In another embodiment of the invention, an actuator wire 14 may be provided extending proximally from the shank 9, with the hard nose 202, to the proximal handle 15. Movement of the proximal handle 15 proximally relative to the distal handle 16 and the catheter body 2 may be limited by a stop means. In this manner the shank 9 with the hard nose 202 may provide a stop to limit proximal movement of the medical device being retrieved into the guidewire lumen 7. This arrangement may therefore eliminate the need for radiopacity in the system.

It will be appreciated that the actuator wire 14 may extend externally of the catheter body 2 along the catheter body 2 from the proximal end of the catheter to the shank 9.

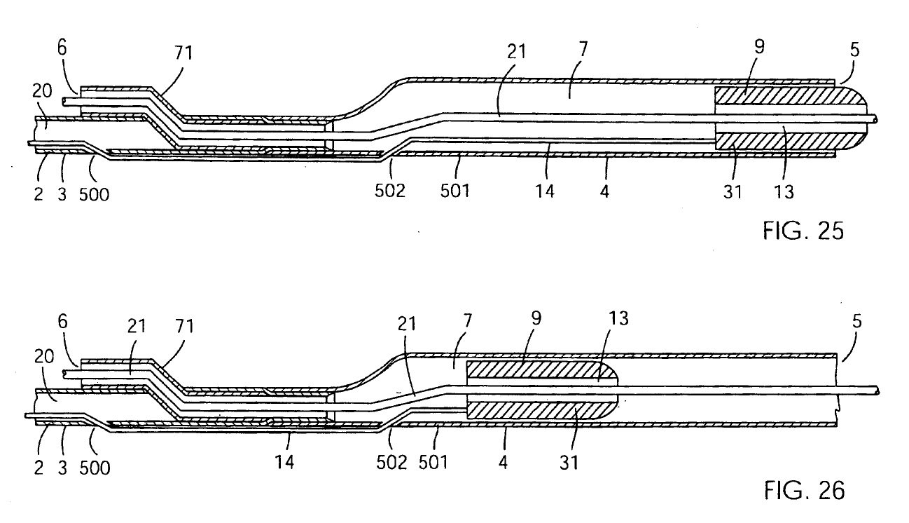

Alternatively, as illustrated in the retrieval catheter 501 of FIGS. 25 and 26, the actuator wire 14 may extend distally through an actuator lumen 20 in the hypotube 3, exit the hypotube 3 through a proximal opening 500 in the sidewall of the hypotube 3, extend externally along the shaft 4, pass into the guidewire lumen 7 through a distal opening 502 in the shaft 4, and extend distally to the shank 9 to which the actuator wire 14 is coupled.

The guide tube 71 assists in guiding passage of the guidewire 21 from the guidewire lumen 7 out through the proximal guidewire opening 6.

The proximal actuator opening 500 is located proximally of the guidewire opening 6, and the distal actuator opening 502 is located distally of the guidewire opening 6.

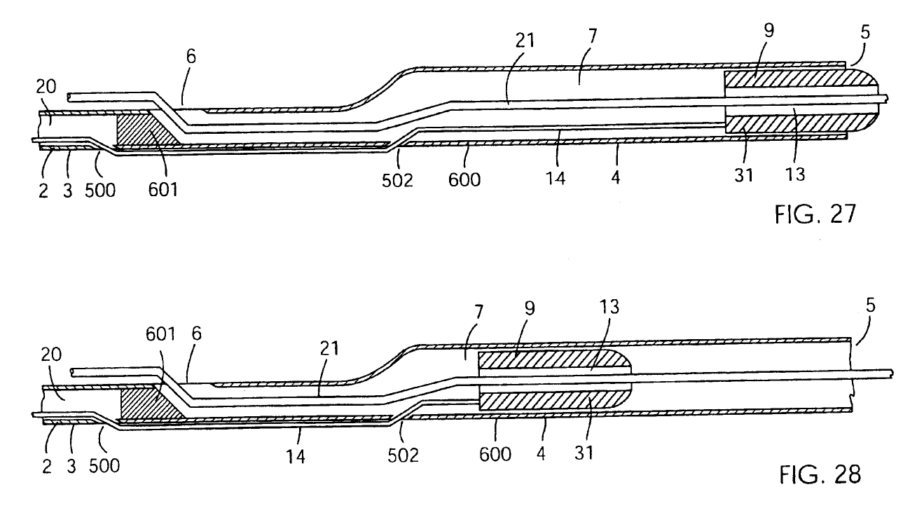

A guide ramp 601 may alternatively be provided at the guidewire opening 6 to assist in guiding passage of the guidewire 21 through the opening 6, as illustrated in the retrieval catheter 600 of FIGS. 27 and 28.

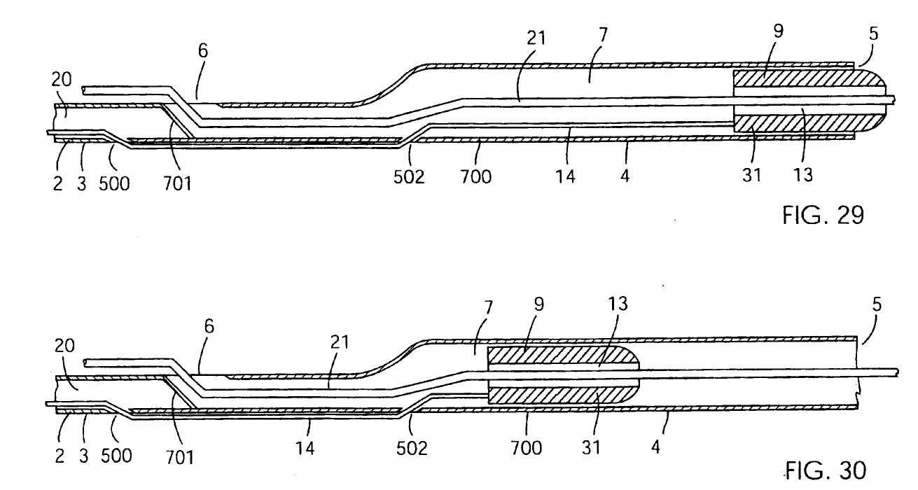

As a further alternative a folded down lip or tongue 701 may be provided at the guidewire opening 6 to assist in guiding passage of the guidewire 21 through the opening 6, as illustrated in the retrieval catheter 700 of FIGS. 29 and 30.

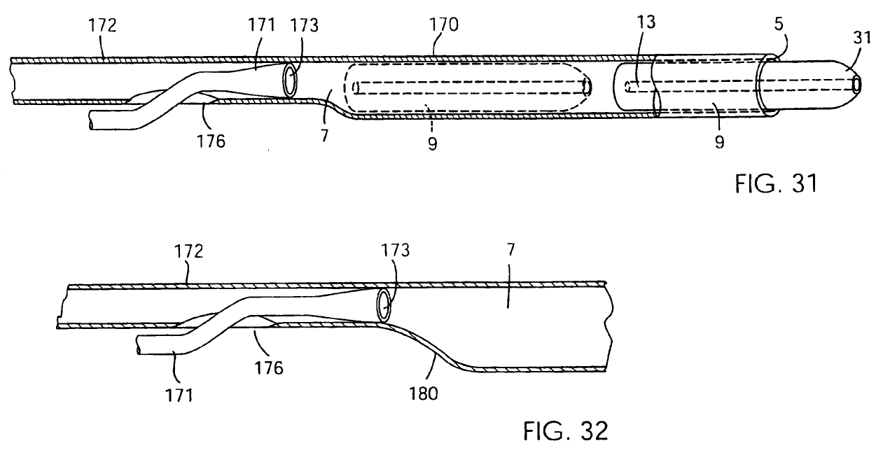

FIG. 31 illustrates another retrieval catheter 70 according to the invention, which is similar to the retrieval catheter 501 of FIGS. 25 and 26, and similar elements in FIG. 31 are assigned the same reference numerals.

The catheter body 2 is provided in this case by a unitary catheter piece 172 extending from the proximal end of the catheter 170 to the distal end of the catheter 170.

The catheter body 2 defines the proximal guidewire opening 176 in a sidewall of the catheter body 2. The guidewire 21 may be passed into the guidewire lumen 13 of the shank 9, through the guidewire lumen 7 of the catheter body 2, and out through the proximal guidewire opening 176 to facilitate rapid exchange of the retrieval catheter 170 over the guidewire 21.

The catheter 170 comprises a guide tube 171, through which the guidewire 21 may pass, to assist passage of the guidewire 21 through the proximal guidewire opening 176. The guide tube 171 is fixed to the catheter body 172 at the opening 176, with a proximal portion of the tube 171 extending proximally externally of the guidewire lumen 7 and a distal portion of the tube 171 extending distally into the guidewire lumen 7.

The tube 171 tapers distally outwardly to an enlarged open mouth 173 through which the guidewire 21 enters the tube 171. The flared open mouth 173 assists in guiding passage of the guidewire 21 into the tube 171 by providing a gradual transition in diameter from the large guidewire lumen 7 of the catheter body 2 to the smaller internal lumen of the tube 171.

The guide tube 171 assists in rapid-exchange of the retrieval catheter 170 over the guidewire 21 in a smooth, controlled manner.

No junction piece is provided in the retrieval catheter 170.

In the retrieval catheter 180 of FIG. 32, the tube 171 does not taper distally outwardly to the open mouth 173. Instead the catheter body 2 tapers inwardly proximally towards the open mouth 173 to assist in guiding passage of the guidewire 21 into the tube 171.

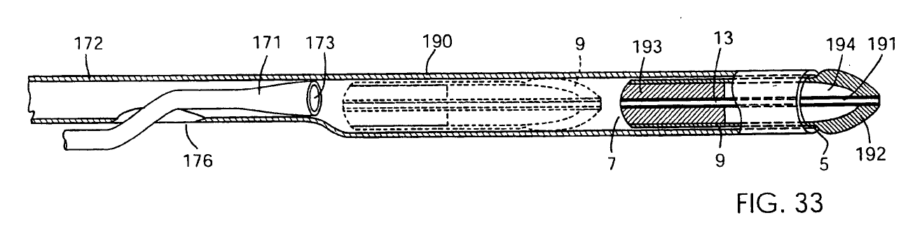

In FIGS. 33 to 36 there is illustrated another retrieval catheter 190 according to the invention, which is similar to the retrieval catheter 170 of FIG. 31, and similar elements in FIGS. 33 to 36 are assigned the same reference numerals.

In this case, the shank 9 comprises a longitudinally stiff inner shaft 191 and a radially compressible outer body 192.

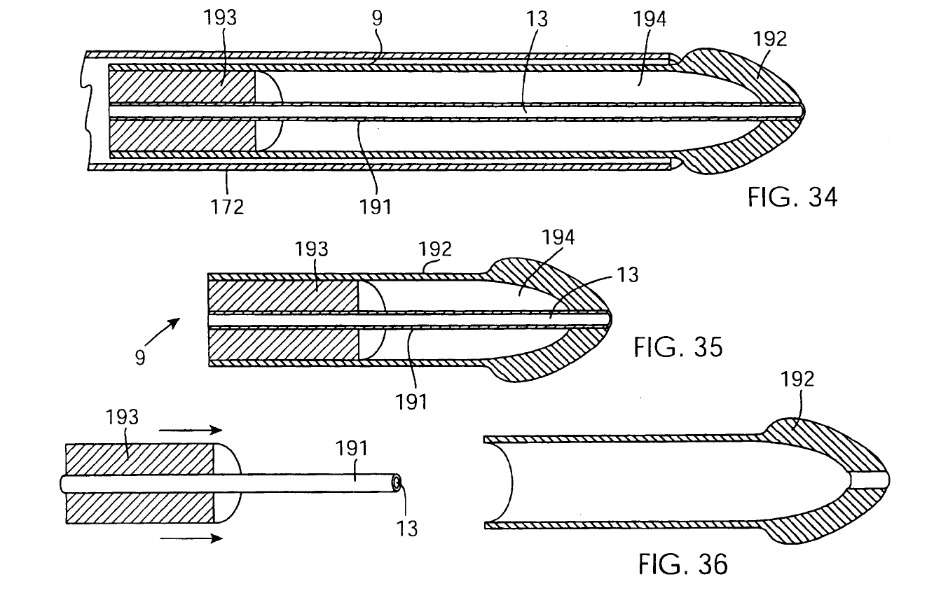

As illustrated in FIG. 34, the outer body 192 has a substantially arrow-head shape in the introduction configuration. This arrangement ensures a smooth crossing profile for the retrieval catheter 190.

The inner shaft 191 defines the guidewire lumen 13 therethrough for passage of the guidewire 21 through the shank 9.

The compressible nature of the outer body 192 facilitates retrieval of the arrow-head shaped shank 9 into the guidewire lumen 7, and the stiff nature of the inner shaft 191 provides longitudinal support for the shank 9.

A suitable material for the outer body 192 is a soft polymeric material.

The outer body 192 is fixedly attached to the inner shaft 191 by means of a cylindrical centering piece 193 at the proximal end of the shank 9, and the outer body 192 is directly attached to the inner shaft 191 at the distal end of the shank 9, as illustrated in FIG. 35.

Between the centering piece 193 and the distal end of the shank 9, the outer body 192 is spaced radially from the inner shaft 191 to define a deflection space 194 (FIG. 35). The deflection space 194 facilitates radially inward deflection of the outer body 192 upon movement of the shank 9 from the introduction configuration to the retrieval configuration.

The shank 9 may be formed by inserting the inner shaft 191 through the outer body 192 (FIG. 36). The distal end of the inner shaft 191 is fixed to the distal end of the outer body 192, for example by means of welding or bonding or fusing. The centering piece 193 is fixed to the proximal end of the inner shaft 191, for example by means of welding or bonding or fusing, and the centering piece 193 is fixed to the proximal end of the outer body 192, for example by means of welding or bonding or fusing.

The centering piece 193 and the inner shaft 191 may alternatively be integrally formed, for example by machining.

It will be appreciated that the shank 9 may be shaped in any suitable manner to achieve a smooth crossing profile for the retrieval catheter of the invention.

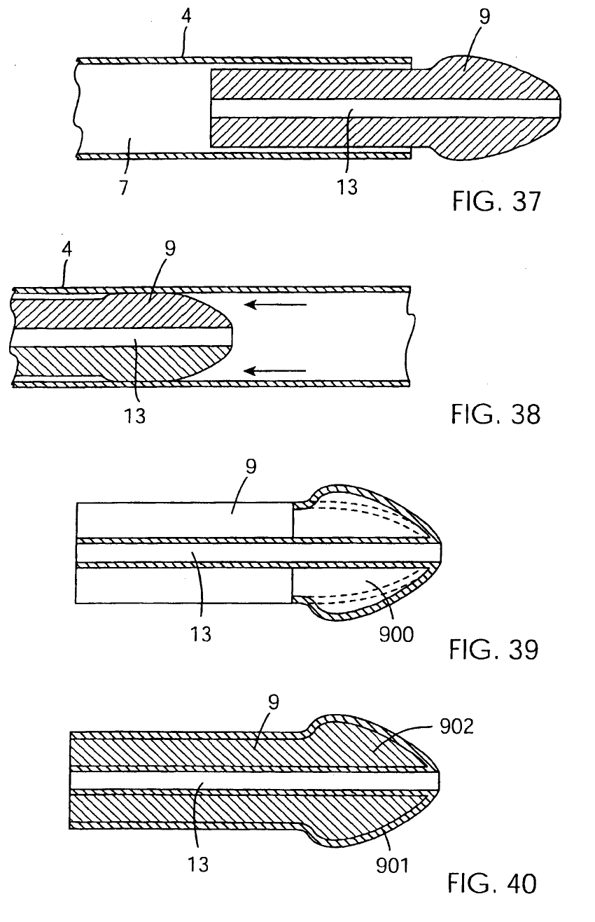

The shank 9 may be of a soft polymeric material shaped in the form of an arrow-head, as illustrated in FIGS. 37 and 38. Upon retraction of the shank 9 into the shaft 4, the shank 9 compresses uniformly to fit within the relatively small diameter guidewire lumen 7 (FIG. 38).

A compression void 900 may be provided in the shank 9, as illustrated in FIG. 39, to enable the shank 9 to at least partially compress to fit within the relatively small diameter guidewire lumen 7 of the shaft 4.

As a further alternative the shank 9 may be of a multi-layer construction. For example, the shank 9 may comprise a relatively hard outer surface 901 and a relatively soft inner layer 902, as illustrated in FIG. 40. The softer inner layer 902 is compressible to enable the shank 9 to fit within the relatively small diameter guidewire lumen 7 of the shaft 4.

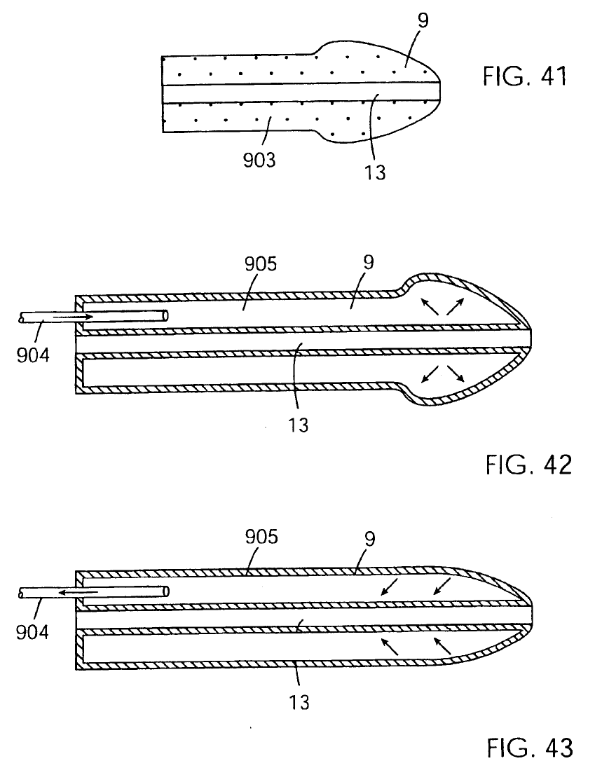

The shank 9 may comprise a porous material 903, such as a cellular foam, as illustrated in FIG. 41, to enable the shank 9 to at least partially compress to fit within the relatively small diameter guidewire lumen 7 of the shaft 4.

As illustrated in FIGS. 42 and 43, the shank 9 may be at least partially inflatable/deflatable to enable the shank 9 to move between a smooth crossing profile arrow-head shape (FIG. 42), and a smaller diameter profile to fit within the relatively small diameter guidewire lumen 7 of the shaft 4. An inflation fluid may be passed into and out of an inflation space 905 in the shank 9 through an inflation tube 904 to inflate (FIG. 42) and deflate (FIG. 43) the shank 9 respectively.

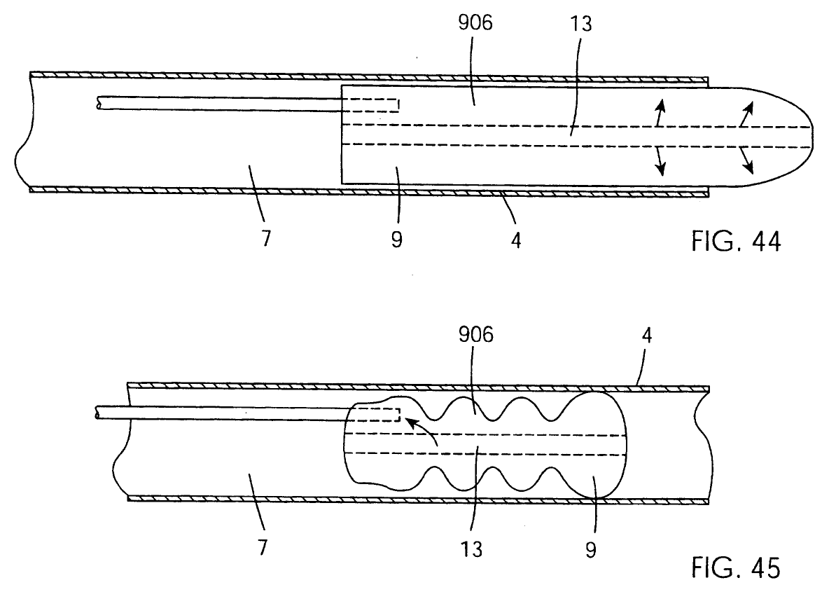

FIGS. 44 and 45 illustrate another inflatable shank 906 similar to the shank 9 described previously with reference to FIGS. 42 and 43.

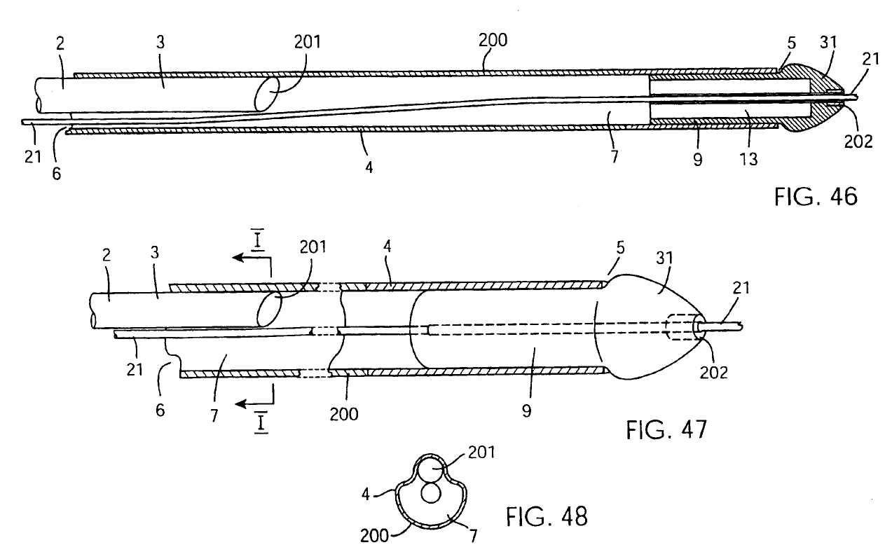

Referring to FIGS. 46 to 48, there is illustrated a further retrieval catheter 200 according to the invention, which is similar to the retrieval catheter 600 of FIGS. 27 and 28, and similar elements in FIGS. 46 to 48 are assigned the same reference numerals.

In this case the shaft 4 is directly fixed to the hypotube 3, for example by means of bonding, or welding, or fusing, with the proximal end of the shaft 4 overlapping the distal end of the hypotube 3.

A distal end face 201 of the hypotube 3 is sloped, and thus acts as a ramp to guide passage of the guidewire 21 through the proximal guidewire opening 6.

Because the shaft 4 partially overlaps the hypotube 3, the distal end face 201 is located distally of the proximal guidewire opening 6. This arrangement enables a longitudinally shorter shank 9 to be used, which minimizes the surface area in contact between the shank 9 and the shaft 4, thus minimizing the frictional losses during relative movement between the shank 9 and the shaft 4.

The shank 9 comprises a distal nose 202 of a relatively hard material at the distal end of the relatively soft polymeric member 31. The nose 202 presents a hard engagement surface against which the filter being retrieved may engage. Thus the softer polymeric member 31 is prevented from becoming wedged onto the filter during the retrieval action.

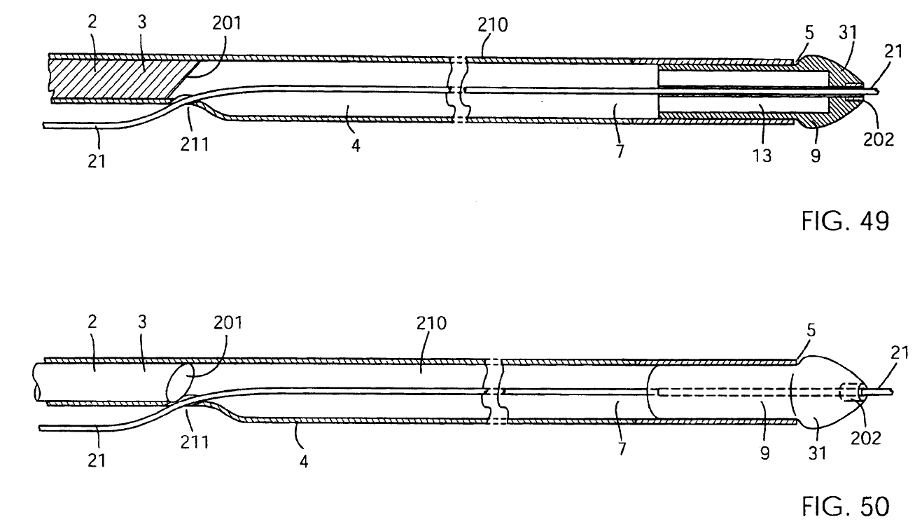

In FIGS. 49 and 50 there is illustrated another retrieval catheter 210 according to the invention, which is similar to the retrieval catheter 200 of FIGS. 46 to 48, and similar elements in FIGS. 49 and 50 are assigned the same reference numerals.

The proximal guidewire opening 211 is provided, in this case, in the form of an opening through the sidewall of the shaft 4, and the sloping distal end face 201 of the hypotube 3 is located substantially adjacent the guidewire opening 211. The distal end face 201 acts as a ramp to guide passage of the guidewire 21 through the proximal guidewire opening 211.

In addition, the shaft 4 tapers proximally inwardly towards the end face 201 and the proximal guidewire opening 211. This tapered arrangement assists in guiding passage of the guidewire 21 through the proximal guidewire opening 211.

It will be appreciated that the relatively hard engagement surface on the relatively soft centering shank may alternatively be used in a retrieval catheter adapted for over-the-wire exchange over a guidewire. In this case, the shank may be provided at a distal end of a centering member, which extends through the catheter body from a proximal end located proximally of the proximal end of the catheter body.

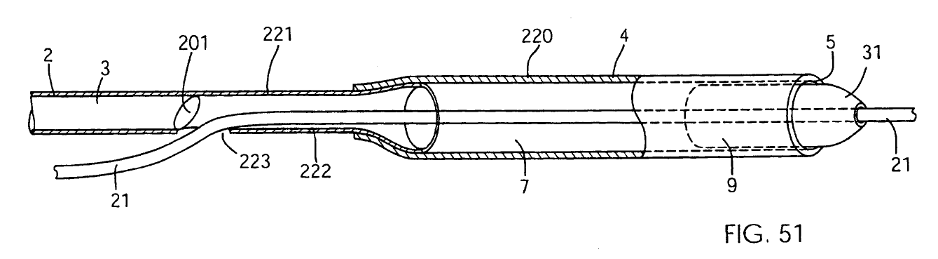

FIG. 51 illustrates a further retrieval catheter 220 according to the invention, which is similar to the retrieval catheter 210 of FIGS. 49 and 50, and similar elements in FIG. 51 are assigned the same reference numerals.

The catheter 220 comprises a hollow transition piece 221 between the hypotube 3 and the sheath 4. The transition piece 221 has a guidewire lumen 222 extending therethrough and the proximal guidewire opening 223 is formed as an opening in the sidewall of the transition piece 221.

The transition piece 221 is fixed to the proximal end of the sheath 4 with the guidewire lumen 222 of the transition piece 221 aligned with the guidewire lumen 7 of the sheath 4 for smooth passage of the guidewire 21 from the sheath 4 into the transition piece 221. The transition piece 221 is fixed to the distal end of the hypotube 3 with the sloping distal end face 201 adjacent the proximal guidewire opening 223. In this way the end face 201 assists in guiding passage of the guidewire 21 through the proximal guidewire opening 223.

The transition piece 221 tapers proximally inwardly from the sheath 4 towards the sloping end face 201 to assist in guiding passage of the guidewire 21 through the proximal guidewire opening 223.

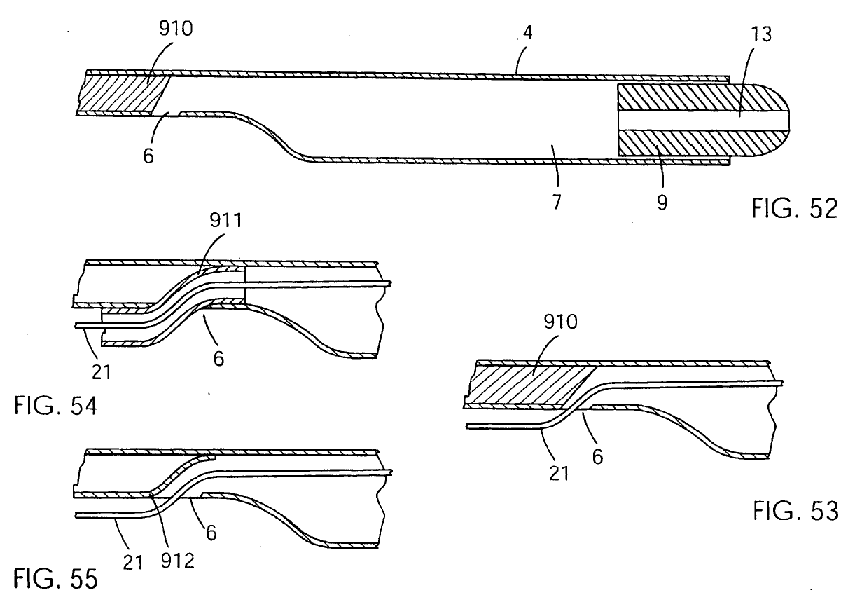

The guiding means to assist passage of the guidewire 21 from the guidewire lumen 7 of the shaft 4 out through the proximal guidewire opening 6 may be provided in the form of a guiding ramp 910 (FIGS. 52 and 53), or a guide tube 911 (FIG. 54), or a tongue or lip to guide the guidewire 21 (FIG. 55).

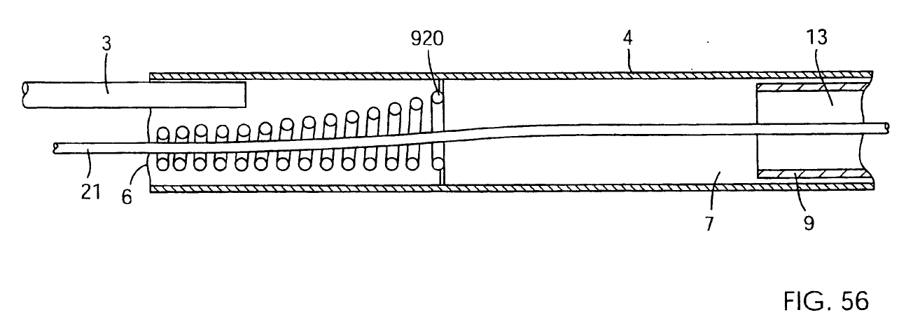

Alternatively a tapered coiled spring 920 may be provided fixed to the shaft 4 at the proximal guidewire opening 6, as illustrated in FIG. 56, to assist guiding of the guidewire 21 from the guidewire lumen 7 out through the proximal guidewire opening 6.

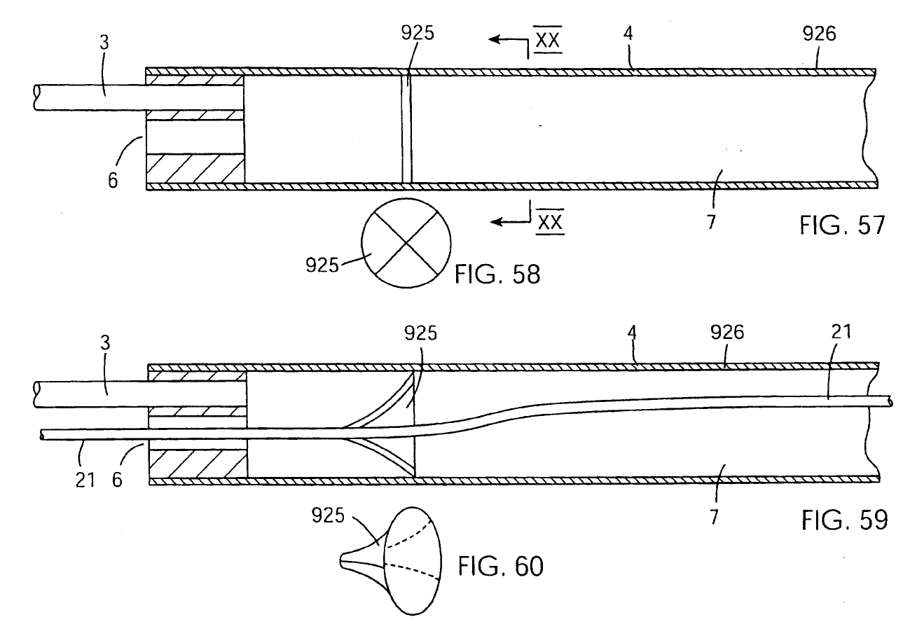

In the retrieval catheter 926 of FIGS. 57 to 60, the catheter 926 comprises four guiding vanes 925 fixed to the sheath 4 distally of the proximal guidewire opening 6. The vanes 925 are deflectable proximally upon passage of the guidewire 21 therethrough, and in this manner the vanes 925 assist in guiding passage of the guidewire 21 towards the proximal guidewire opening 6.

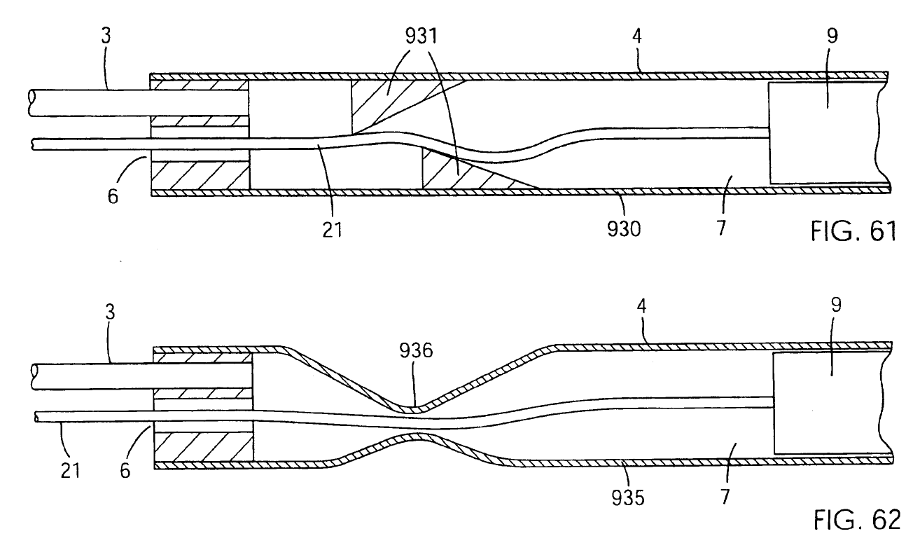

In the retrieval catheter 930 of FIG. 61, guiding baffles 931 are provided to center the guidewire 21 and thus assist in guiding passage of the guidewire 21 through the proximal guidewire opening 6.

In the retrieval catheter 935 of FIG. 62, a necked down portion 936 of the shaft 4 centers the guidewire 21 and thus assists in guiding passage of the guidewire 21 through the proximal guidewire opening 6.

It will be appreciated that any of the various guide means described previously, such as the junction piece, and/or the guide tube, and/or the telescoping guide tubes, and/or the sloping distal end face, and/or the tapered section of the catheter body, may be employed with any rapid exchange catheter to control passage of the guidewire through a guidewire lumen of the catheter and/or through a guidewire exit port. A centering means may or may not be used with the rapid exchange catheter.

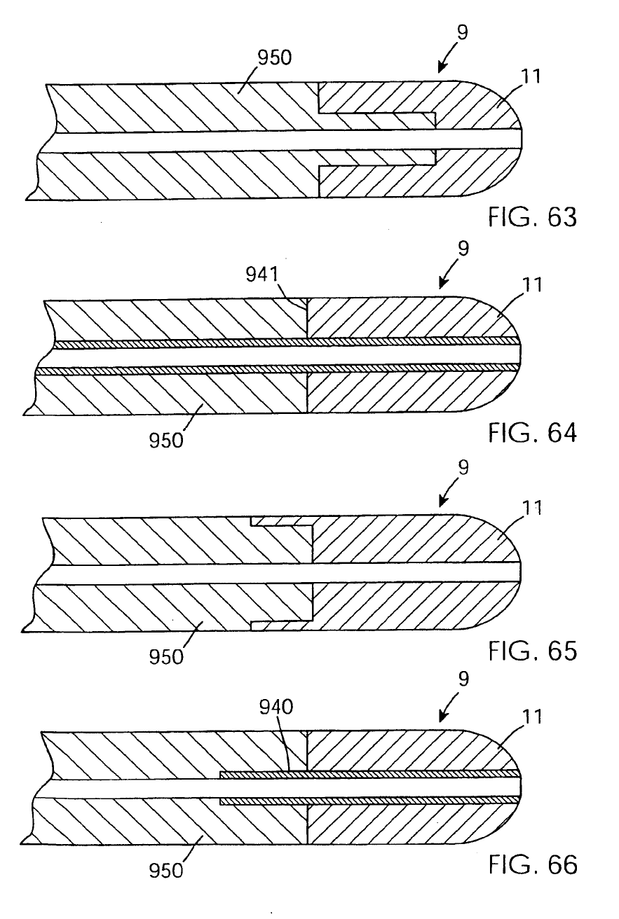

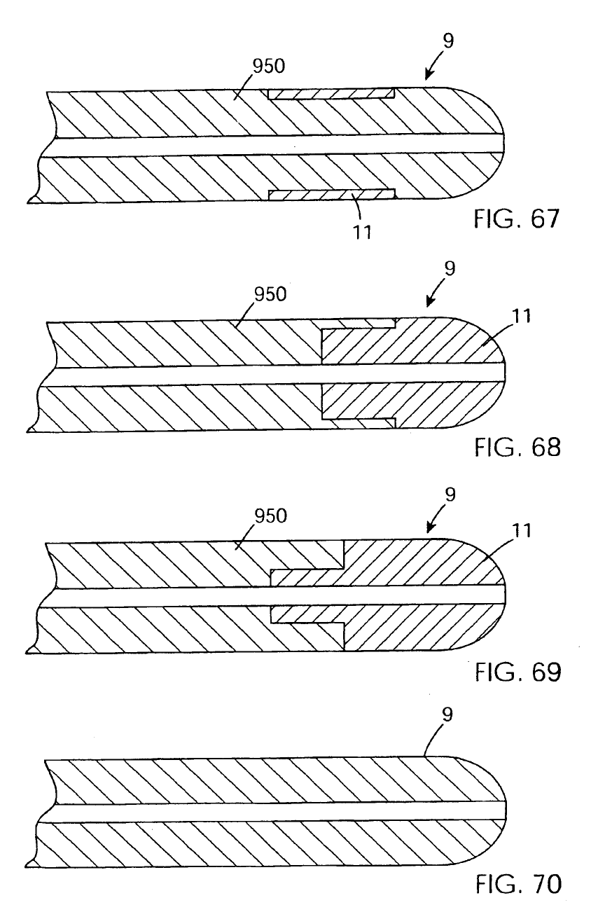

As illustrated in FIGS. 63 to 69, the distal compressible tip 11 of the shank 9 may be fixedly attached to the main portion 950 of the shank 9 by any suitable geometric keying means. A butt weld 941 is used in FIG. 64, and a keying bridge 940 is provided in the shank 9 of FIG. 66.

In another case the distal compressible tip may be integrally formed with the main portion of the shank 9, as illustrated in FIG. 70.

The centering means may alternatively be located in a non co-axial relationship relative to the catheter body and/or the guidewire.

The ability to control the centering element during the procedure provides the user with a number of safety and handling benefits. The ability of the user to retract the tip separate of the retrieval action reduces the force of retrieval and improves the feel. This improved feel allows the clinician to detect issues such as filter snagging on stent struts and the like. This ability provides important patient safety benefits.

The invention is not limited to the embodiments hereinbefore described, with reference to the accompanying drawings, which may be varied in construction and detail.

What is claimed is:

1. A retrieval catheter comprising:
a catheter shaft defining a reception space for a medical device;
a centering member movable relative to the catheter shaft between an introduction configuration in which the centering member extends distally of the catheter shaft, and a retrieval configuration in which the centering member is located proximally of a distal end of the catheter shaft for retrieval of a medical device into the reception space; and
an operating element coupled to the centering member to facilitate movement of the centering member between the introduction configuration and the retrieval configuration;
along at least a portion of the length of the operating element, the cross-sectional area of the operating element being small relative to the cross-sectional area of the catheter shaft.

2. A catheter as claimed in claim 1, wherein a guidewire opening is provided in the catheter shaft, the guidewire opening being located a substantial distance distally of a proximal end of the catheter for rapid exchange of the catheter over a guidewire.

3. A catheter as claimed in claim 2, wherein the cross-sectional area of the operating element is small relative to the cross-sectional area of the catheter shaft in the region of the guidewire opening.

4. A catheter as claimed in claim 3, wherein in the introduction configuration the cross-sectional area of the operating element is small relative to the cross-sectional area of the catheter shaft for a distance of at least 10 mm distally of the guidewire opening.

5. A catheter as claimed in claim 4, wherein in the introduction configuration the cross-sectional area of the operating element is small relative to the cross-sectional area of the catheter shaft for a distance of at least 20 mm distally of the guidewire opening.

6. A catheter as claimed in claim 5, wherein in the introduction configuration the cross-sectional area of the operating element is small relative to the cross-sectional area of the catheter shaft for a distance of at least 30 mm distally of the guidewire opening.

7. A catheter as claimed in claim 6, wherein in the introduction configuration the cross-sectional area of the operating element is small relative to the cross-sectional area of the catheter shaft for a distance of at least 40 mm distally of the guidewire opening.

8. A catheter as claimed in claim 1, wherein the diameter of the operating element is in the range of from 0.008″ to 0.015″.

9. A catheter as claimed in claim 8, wherein the diameter of the operating element is in the range of from 0.01″ to 0.012″.

10. A catheter as claimed in claim 1, wherein in the retrieval configuration the centering member is located in the reception space.

11. A catheter as claimed in claim 10, wherein in the retrieval configuration the centering member is configured to fit in the reception space distally of the guidewire opening.

12. A catheter as claimed in claim 1, wherein the centering member is slidably movable relative to the catheter shaft between the introduction configuration and the retrieval configuration.

13. A catheter as claimed in claim 1, wherein the centering member comprises a coiled spring.

14. A catheter as claimed in claim 1, wherein the centering member comprises longitudinal stiffening means.

15. A catheter as claimed in claim 14, wherein the longitudinal stiffening means comprises a shaft extending through at least part of the centering member.

16. A catheter as claimed in claim 1, wherein the centering member comprises an atraumatic distal tip.

17. A catheter as claimed in claim 16, wherein the tip tapers distally inwardly.

18. A catheter as claimed in claim 1, wherein the centering member defines a guidewire lumen therethrough.

19. A catheter as claimed in claim 18, wherein the guidewire lumen of the centering member is movable relative to the guidewire opening in the catheter shaft upon movement of the centering member between the introduction configuration and the retrieval configuration.

20. A catheter as claimed in claim 1, wherein the operating element is attached to the centering member.

21. A catheter as claimed in claim 20, wherein the operating element is integral with at least part of the centering member.

22. A catheter as claimed in claim 20, wherein the operating element is rotatably attached to the centering member.

23. A catheter as claimed in claim 22, wherein the operating element comprises a ring rotatably received in a groove on the centering member.

24. A catheter as claimed in claim 20, wherein the centering member extends distally of the operating element.

25. A catheter as claimed in claim 1, wherein the operating element comprises a control wire.

26. A catheter as claimed in claim 25, wherein the operating element comprises a pull wire.

27. A catheter as claimed in claim 1, wherein the operating element comprises a coiled spring.

28. A catheter as claimed in claim 1, wherein the operating element comprises a fiber.

29. A catheter as claimed in claim 1, wherein the operating element comprises a hypotube.

30. A catheter as claimed in claim 1, wherein the operating element is at least partially of a polymeric material.

31. A catheter as claimed in claim 1, wherein the operating element defines a lumen therethrough.

32. A catheter as claimed in claim 1, wherein the catheter comprises two or more operating elements.

33. A catheter as claimed in claim 32, wherein the operating elements are braided together.

34. A catheter as claimed in claim 1, wherein the catheter shaft comprises a proximal shaft portion and a distal pod, the pod at least partially defining the reception space.

35. A catheter as claimed in claim 34, wherein the proximal shaft portion is offset in the radial direction from the pod.

36. A catheter as claimed in claim 34, wherein the proximal shaft portion is of a smaller diameter than the pod.

37. A catheter as claimed in claim 34, wherein the catheter shaft comprises a mounting piece for attaching the pod to the proximal shaft portion.

38. A catheter as claimed in claim 37, wherein the distal end of the proximal shaft portion is located distally of the proximal end of the pod.

39. A catheter as claimed in claim 38, wherein the mounting piece is more flexible than the proximal shaft portion and the pod.

40. A catheter as claimed in claim 37, wherein the mounting piece is more stiff than the proximal shaft portion and the pod.

41. A catheter as claimed in claim 37, wherein the mounting piece tapers proximally inwardly.

42. A catheter as claimed in claim 37, wherein the mounting piece tapers distally inwardly.

43. A catheter as claimed in claim 37, wherein the guidewire opening in the catheter shaft is provided by an opening in the mounting piece.

44. A catheter as claimed in claim 2, wherein the guidewire opening faces in a direction substantially parallel to the longitudinal axis of the catheter.

45. A catheter as claimed in claim 44, wherein the guidewire opening faces proximally.

46. A catheter as claimed in claim 2, wherein the catheter shaft comprises means to guide passage of a guidewire through the guidewire opening in the catheter shaft.

47. A catheter as claimed in claim 46, wherein the guide means comprises a guide ramp.

48. A catheter as claimed in claim 47, wherein the ramp is funnel-shaped.

49. A catheter as claimed in claim 46, wherein the guide means comprises at least one guide tube through which a guidewire may pass.

50. A catheter as claimed in claim 49, wherein the guide tube extends at least partially internally through the catheter shaft.

51. A catheter as claimed in claim 49, wherein the guide tube extends at least partially externally of the catheter shaft.

52. A catheter as claimed in any of claims 49 to 51, wherein the guide tube is mounted to the catheter shaft.

53. A catheter as claimed in claim 49, wherein the guide tube is mounted to the centering member.

54. A catheter as claimed in claim 49, wherein the catheter comprises two or more guide tubes movable relative to one another in a telescoping manner.

55. A retrieval catheter for retrieving an embolic protection filter from a vasculature as claimed in claim 1.

56. A retrieval catheter for retrieving a stent from a vasculature as claimed in claim 1.

57. A catheter comprising a proximal shaft portion and a distal shaft portion attached to the proximal shaft portion, and means to stiffen the catheter at the junction between the proximal shaft portion and the distal shaft portion.

58. A catheter as claimed in claim 57, wherein the catheter comprises a mounting piece for attaching the distal shaft portion to the proximal shaft portion.

59. A catheter as claimed in claim 57, wherein the distal end of the proximal shaft portion is located distally of the proximal end of the distal shaft portion to stiffen the junction.

60. A catheter as claimed in claim 59, wherein the mounting piece is more flexible than the proximal shaft portion and the distal shaft portion.

61. A catheter as claimed in claim 58, wherein the mounting piece is more stiff than the proximal shaft portion and the distal shaft portion to stiffen the junction.

62. A catheter as claimed in claim 57, wherein the catheter comprises strain relief means.

63. A catheter as claimed in claim 62, wherein the mounting piece tapers distally inwardly.

64. A catheter as claimed in claim 62, wherein the mounting piece tapers proximally inwardly.

65. A catheter as claimed in claim 57, wherein a guidewire opening is provided in the catheter, the guidewire opening being located a substantial distance distally of a proximal end of the catheter for rapid exchange of the catheter over a guidewire.

66. A catheter as claimed in claim 65, wherein the guidewire opening is provided by an opening in the mounting piece.

67. A catheter as claimed in claim 65, wherein the guidewire opening faces in a direction substantially parallel to the longitudinal axis of the catheter.

68. A catheter as claimed in claims 65, wherein the catheter comprises means to guide passage of a guidewire through the guidewire opening in the catheter.

69. A catheter as claimed in claim 68, wherein the means to guide passage is provided by the mounting piece.

70. A retrieval catheter comprising:
a catheter shaft defining a reception space for a medical device;
a guidewire opening being provided in the catheter shaft; and
a centering member movable relative to the catheter shaft between an introduction configuration in which the centering member extends distally of the catheter shaft, and a retrieval configuration in which the centering member is located proximally of a distal end of the catheter shaft for retrieval of a medical device into the reception space;
the centering member defining a guidewire lumen therethrough;
the guidewire lumen of the centering member being movable relative to the guidewire opening in the catheter shaft upon movement of the centering member between the introduction configuration and the retrieval configuration.

71. A catheter as claimed in claim 70, wherein the guidewire opening in the catheter shaft is located a substantial distance distally of a proximal end of the catheter for rapid exchange of the catheter over a guidewire.

72. A catheter as claimed in claim 70, wherein the guidewire opening in the catheter shaft faces in a direction substantially parallel to the longitudinal axis of the catheter.

73. A catheter as claimed in claim 70, wherein the catheter comprises means to guide passage of a guidewire through the guidewire opening in the catheter shaft.

74. A catheter as claimed in claim 70, wherein the catheter comprises an operating element coupled to the centering member to facilitate movement of the centering member between the introduction configuration and the retrieval configuration.

75. A catheter as claimed in claim 70, wherein the centering member is slidably movable relative to the catheter shaft.

76. A retrieval catheter comprising:
a catheter shaft defining a reception space for a medical device;
a guidewire opening being provided in the catheter shaft located a substantial distance distally of a proximal end of the catheter for rapid exchange of the catheter over a guidewire;
a centering member movable relative to the catheter shaft between an introduction configuration in which the centering member extends distally of the catheter shaft, and a retrieval configuration in which the centering member is located proximally of a distal end of the catheter shaft for retrieval of a medical device into the reception space; and
an operating element coupled to the centering member to facilitate movement of the centering member between the introduction configuration and the retrieval configuration;
a distal end of the centering member and a proximal end of the centering member translating relative to the catheter shaft during movement of the centering member relative to the catheter shaft.

77. A catheter as claimed in claim 76, wherein along at least a portion of the length of the operating element, the cross-sectional area of the operating element is small relative to the cross-sectional area of the catheter shaft.

78. A catheter as claimed in claim 76, wherein in the retrieval configuration the centering member is located in the reception space.

79. A method of retrieving a medical device from a vasculature, the method comprising the steps of:
providing a retrieval catheter comprising a catheter shaft and a centering member;
advancing the catheter through a vasculature over a guidewire in a rapid-exchange manner, with the centering member extending from a distal end of the catheter shaft, until the centering member is proximally adjacent to the medical device to be retrieved;
manipulating the centering member from externally of the vasculature to move the centering member proximally relative to the catheter shaft;
retrieving the medical device into the catheter shaft; and
withdrawing the retrieval catheter and the retrieved medical device from the vasculature over the guidewire in a rapid exchange manner.

80. A method as claimed in claim 79, wherein the guidewire is advanced through the vasculature before inserting the retrieval catheter into the vasculature.

81. A method as claimed in claim 79, wherein the guidewire remains in the vasculature during withdrawal of the retrieval catheter from the vasculature.
